(19) United States
(12) Patent Application Publication
(10) Pub. No.: US 2006/0134693 A1
(43) Pub. Date: Jun. 22, 2006

(54) Olfactory cyclic nucleotide-gated channel cell-based assays to identify T1R and T2R taste modulators

(76) Inventors: Guy Servant, San Diego, CA (US); Mark Ozeck, San Diego, CA (US); Paul Brust, San Diego, CA (US); Hong Xu, San Diego, CA (US); Lena Staszewski, San Diego, CA (US); Jon Elliot Adler, San Diego, CA (US); Mark Zoller, La Jolla, CA (US); Nicholas Callamaras, San Diego, CA (US); Alexey Pronin, San Diego, CA (US); Bryan Moyer, San Diego, CA (US)

Correspondence Address: DUANE MORRIS LLP, 1667 K. STREET, N.W., SUITE 700, WASHINGTON, DC, 20006-1608, US

(21) Appl. No.: 11/006,802

(22) Filed: Dec. 8, 2004

Related U.S. Application Data
(63) Continuation-in-part of application No. 10/770,127, filed on Feb. 3, 2004, now Pat. No. 7,022,488.
(63) Continuation-in-part of application No. 10/189,507, filed on Jul. 8, 2002.
(60) Provisional application No. 60/457,318, filed on Mar. 26, 2003.
(60) Provisional application No. 60/444,172, filed on Feb. 3, 2003.
(60) Provisional application No. 60/303,140, filed on Jul. 6, 2001.
(60) Provisional application No. 60/337,151, filed on Dec. 10, 2001.

(51) Int. Cl. G01N 33/53; C07K 14/705

(52) U.S. Cl. 435/7.1; 530/350

(57) ABSTRACT

Screening assays, preferably high throughput, are provided that screen libraries of candidate compounds to identify agonists, antagonists, enhancers or modulators of taste receptors (bitter, sweet or savory (umami) taste receptor) using test cells that co-express at least one functional taste receptor and an olfactory cyclic nucleotide-gated channel (oCNGC). (The oCNGC preferably comprises at least one mutation in one or more subunits that renders the resultant oCNGC more sensitive to CAMP (which in turn enhances the sensitivity of assay using this oCNGC). These taste modulatory compounds are identified based on their effect on oCNGC activity, e.g., using fluorimetric assays that screen for changes in intracellular calcium or sodium concentration in test cells that co-express at least one taste receptor, oCNGC and a G_{αi/o} protein.

### RELATED APPLICATIONS

This application is a continuation-in-part of U.S. Ser. No. 10/770,127 filed Feb. 3, 2004, which claims benefit of priority to U.S. Provisional Ser. No. 60/457,318 filed Mar. 26, 2003 and to U.S. Ser. No. 60/444,172 filed on Feb. 3, 2003. Additionally, this application is a continuation-in-part of U.S. Ser. No. 10/189,507 filed Jul. 8, 2002, which claims priority to Provisional Application Ser. No. 60/303,140 filed Jul. 6, 2001 and to Provisional Ser. No. 60/337,151 filed Dec. 10, 2001. All of these related applications are incorporated by reference in their entireties herein.

### FIELD OF THE INVENTION

The present invention relates to novel methods and materials for the identification of modulators, e.g., enhancers, modulators of G protein-coupled receptors (GPCRs) involved in taste, i.e., T1Rs and T2Rs. These modulators may be used as flavor-affecting additives, e.g., in foods, beverages and medicines for human or animal consumption.

Particularly, the invention provides cell-based assays, preferably high throughput assays that rely in part on Applicants' earlier discovery that G proteins other than gustducin and promiscuous and pernicious G proteins such as Gα₁₅, i.e., Gᵢ proteins, functionally couple to T1Rs and T2Rs.

More particularly, the present invention involves the discovery that agonists, antagonists, enhancers or modulators of specific T1R or T2R taste receptors can be identified by screening their effect on the activity of an olfactory cyclic nucleotide gated channel (oCNGC) comprised in a cell or cell membrane that co-expresses a said oCNGC and a taste receptor, preferably a T1R or T2R.

### BACKGROUND OF THE INVENTION

The family of receptors that transmit signals through the activation of heterotrimeric GTP-binding proteins (G proteins) constitutes the largest group of cell surface proteins involved in signal transduction. These receptors participate in a broad range of important biological functions and are implicated in a number of disease states. More than half of all drugs currently available influence GPCRs. These receptors affect the generation of small molecules that act as intracellular mediators or second messengers, and can regulate a highly interconnected network of biochemical routes controlling the activity of several members of the mitogen-activated protein kinase (MAPK) superfamily.

In fact, the activation of members of the mitogen-activated protein kinase (MAPK) family represents one of one of the major mechanisms used by eukaryotic cells to transduce extracellular signals into cellular responses (J. Blenis, Proc. Natl. Acad. Sci., USA 90:5889 (1993); Blumer et al., TIBS 19:236 (1994); Cano et al., TIBS 20:117 (1995); Seger et al., FASEB J. 9:726 (1995): R. J. Davis, TIBS 19:470 (1994)). The MAPK superfamily consists of the p42 (ERK2)/p44 (ERK1) MAPKs and the stress-activated protein kinases, c-Jun N-terminal kinase (JNK) and p38 MAPK. (Robinson and Dickenson, Eur. J. Pharmacol. 413(2-3):151-61 (2001)).

Mitogen-activated protein kinase (MAPKs) (also called extracellular signal-regulated kinases or ERKs) are rapidly activated in response to ligand binding by both growth factor receptors that function as tyrosine kinases (such as the epidermal growth factor (EGF) receptor) and receptors that are complexed with heterodimeric guanine nucleotide binding proteins (G proteins) such as the thrombin receptor. In addition, receptors such as the T cell receptor (TCR) and B cell receptor (BCR) are non-covalently associated with src family tyrosine kinases which activate MAPK pathways. Specific cytokines like tumor necrosis factor (TNFalpha) can also regulate MAPK pathways. The MAPKs appear to integrate multiple intracellular signals transmitted by various second messengers. MAPKs phosphorylate and regulate the activity of enzymes and transcription factors including the EGF receptor, Rsk 90, phospholipase A₂, c-Myc, c-Jun and EIK-1/TCF. Although the rapid activation of MAPKs by tyrosine kinase receptors is dependent on Ras, G protein-mediated activation of MAPK also occurs through pathways dependent and independent of Ras.

Particularly, it is known that the activation of MAP/ERK kinase which is induced by GPCRs involves both of the G alpha and G beta gamma subunits and further involves a common signaling pathway with receptor-tyrosine-kinases. (Lopez-llasaca, Biochem. Pharmacol. 56(3): 269-77 (1998)). For example, the G protein beta gamma subunit has been shown to activate Ras, Raf and MAP kinase in HEK-293 cells. (Ito et al., FEBS Lett. 368(1): 183-7 (1995)).

Also of relevance to the present invention is Applicants' previous discovery relating to the cloning and identification of specific human olfactory cyclic nucleotide gated (CNG) channel subunit nucleic acid and polypeptide sequences, the expression thereof in recombinant host cells, particularly HEK-293 cells to produce functional CNG channels, and the use of cell lines that express functional human olfactory CNG channels in assays, particularly high throughput assays to identify compounds that modulate the CNG human olfactory CNG channel activity. This discovery is disclosed in U.S. Ser. No. 10/189,507 filed Jul. 8, 2002, incorporated by reference in its entirety herein. This patent application discloses cell-based assays, in particular cell-based assays that monitor human olfactory CNG channel activity by detecting changes in calcium levels using calcium sensitive fluorescent dyes and fluorescence plate readers or voltage imaging plate readers.

Additionally of relevance to the present invention, the present invention relates to identifying modulators of two types of taste receptors which have been cloned and functionalized within the last five years. During this time, a number of different research groups including the present assignee Senomyx Inc., have reported the identification and cloning of genes from these two GPCR families (T1Rs and T2Rs) that are involved in taste modulation and have obtained experimental results that provide a greater understanding of taste biology. These results indicate that bitter, sweet and amino acid taste, also referred as umami taste, are triggered by activation of two types of specific receptors located at the surface of taste receptor cells (TRCs) on the tongue i.e., T2Rs and T1Rs. (Gilbertson et al., Corr. Opin. Neurobiol., 10(4):519-27 (2000); Margolskee, R F, J. Biol. Chem. 277(1):1-4 (2002); Montmayeur et al., Curr. Opin. Neurobiol., 12(4):366-71 (2002)). It is currently believed that at least 26 and 33 genes encode functional receptors (T2Rs) for bitter tasting substances in human and rodent respectively (Montmayeur et al., Curr. Opin. Neurobiol., 12(4):366-71 (2002); Adler et al., Cell 100(6):693-702 (2000); Matsunami et al., Nature 404(6678):601-4 (2000)). By contrast there are only 3 T1Rs, T1R1, T1R2 and T1R3, which are involved in umami and sweet taste (Li et al., Proc. Natl Acad Sci., USA 99(7):4692-6 (2002); Nelson et al., Nature (6877):199-202 (2002); Nelson et al., Cell 106(3):381-96 (2001)). Structurally, the T1R and T2R receptors possess the hallmark of G protein-coupled receptors (GPCRs), i.e., 7 transmembrane domains flanked by small extracellular and intracellular amino- and carboxyl-termini respectively.

T2Rs which have been cloned from different mammals including rats, mice and humans (Adler et al., Cell 100(6): 611-8 (2000)). T2Rs comprise a novel family of human and rodent G protein-coupled receptors that are expressed in subsets of taste receptor cells of the tongue and palate epithelia. These taste receptors are organized in clusters in taste cells and are genetically linked to loci that influence bitter taste. The fact that T2Rs modulate bitter taste has been demonstrated in cell-based assays. For example, mT2R-5, hT2R-4 and mT2R-8 have been shown to be activated by bitter molecules in in vitro gustducin assays, providing experimental proof that T2Rs function as bitter taste receptors. (Chandrasheker et al., Cell 100(6): 703 (2000)).

The present assignee, Senomyx Inc., has filed a number of patent applications relating to various T2R genes and the corresponding polypeptides and their use in assays, preferably high-throughput cell-based assays for identifying compounds that modulate the activity of T2Rs. These Senomyx applications i.e., U.S. Ser. No. 09/825,882, filed on Apr. 5, 2001, U.S. Ser. No. 191,058 filed Jul. 10, 2002 and U.S. Provisional Application Ser. No. 60/398,727, filed on Jul. 29, 2002 all incorporated by reference in their entireties herein. Additionally, the present assignee has exclusively licensed patent applications relating to T2R genes which were filed by the University of California i.e., U.S. Ser. No. 09/393,634, filed on Sep. 10, 1999, new U.S. Pat. No. 6,558,910 and U.S. Ser. No. 09/510,332, filed Feb. 22, 2000 (recently allowed), that describe various mouse, rat and human T2R sequences and the use thereof in assays for identifying molecules that modulate specific T2Rs and which modulate (enhance or block) bitter taste. These applications and the sequences contained therein are also incorporated by reference in their entireties herein.

Further, the present assignee and its exclusive licensor, the University of California, have both filed a number of patent applications relating to human and rodent T1R taste receptors. Specifically, Senomyx has filed patent applications Ser. No. 09/897,427, filed on Jul. 3, 2001, U.S. Ser. No. 10/179,373, filed on Jun. 26, 2002, and U.S. Ser. No. 09/799,629, filed on Mar. 7, 2001, relating to human T1Rs and their use in assays for identifying, sweet and umami taste modulators. These applications and the sequences contained therein are incorporated by reference in their entirety herein. Additionally, the University of California has filed a number of applications exclusively licensed by Senomyx including U.S. Ser. No. 09/361,631, filed Jul. 27, 1999, now U.S. Pat. No. 6,383,778, issued on May 7, 2002 and U.S. Ser. No. 09/361,652, filed on Jul. 27, 1999, which relates to cloned rat, mouse and human T1R1 and T1R2 genes and the use of the genes and corresponding polypeptides to identify T1R modulators. These University of California applications and the sequences contained therein are also incorporated by reference in their entirety herein.

The three T1R gene members T1R1, T1R2 and T1R3 form functional heterodimers that specifically recognize sweeteners and amino acids (Li et al., Proc. Natl Acad Sci., USA 99(7):4692-6 (2002); Nelson et al., Nature (6877):199-202 (2002); Nelson et al., Cell 106(3):381-96 (2001)). Functional studies performed in HEK-293 cells expressing the promiscuous G protein Gα_{15/16}, also disclosed therein have shown that the rodent and human T1R2/T1R3 combination recognizes natural and artificial sweeteners (Li et al., Proc. Natl Acad Sci., USA 99(7):4692-6 (2002); Nelson et al., Nature (6877):199-202 (2002); Nelson et al., Cell 106(3):381-96 (2001)) while the rodent and human T1R1/T1R3 combination recognizes several L-amino acids and monosodium glutamate (MSG), respectively (Li et al., Proc. Natl Acad Sci., USA 99(7):4692-6 (2002); Nelson et al., Nature (6877):199-202 (2002)). These results, demonstrate that T1Rs are involved in sweet and umami taste.

Particularly, the co-expression of T1R1 and T1R3 in recombinant host cells results in a hetero-oligomeric taste receptor that responds to umami taste stimuli. Umami taste stimuli include by way of example monosodium glutamate and other molecules that elicit a “savory” taste sensation. By contrast, the co-expression of T1R2 and T1R3 in recombinant host cells results in a hetero-oligomeric sweet taste receptor that responds to both naturally occurring and artificial sweeteners. As with T2Rs, T1R DNAs and the corresponding polypeptides have significant application in cell and other assays, preferably high throughput assays, for identifying molecules that modulate T1R taste receptors; particularly the T1R2/T1R3 receptor (sweet receptor) and the T1R1/T1R3 receptor (umami receptor). T1R modulators can be used as flavor-affecting additives in foods, beverages and medicines.

The patents and patent application referenced above, which are incorporated by reference in their entirety herein, disclose a number of assay methods, including cell-based high throughput screening assays for identifying T1R and T2R modulators. (As defined infra, modulators according to the invention include agonists, antagonists and enhancers.) However, notwithstanding what is disclosed therein, novel and improved assays for identifying T1R and T2R modulators are still needed. In particular, other high throughput assays that provide for the rapid and accurate identification of T1R or T2R modulators would be beneficial. Also, a greater understanding of what conditions and materials yield functional T1Rs and T2Rs and assays based on this greater understanding would further be beneficial.

### OBJECTS OF THE INVENTION

Toward that end, it is an object of the invention to provide a greater understanding of the means by which T1Rs and T2Rs functionally couple to G proteins and their signaling pathways.

More particularly, it is an object of the invention to provide further evidence that G proteins other than Gα₁₅ and gustducin (Gα_{i/o} proteins) which functionally couple to GPCRs involved in taste, i.e., T1Rs and T2Rs.

It is specifically an object of the invention to provide assays, preferably cell-based assays which exploit the discovery that T1Rs and T2Rs functionally couple to Gα_{i/o} proteins, e.g., Gαᵢ.

Particularly, it is an object of the invention to provide cell-based assays for identifying T1R and T2R modulators that use techniques which assay the effect of putative modulators on Gα_{i/o} signaling pathways.

It is a more specific object of the present invention to provide cell-based assays for identifying taste receptor modulators, e.g., T1R and T2R modulators that use cell-based techniques which assay the effect of a putative T1R or T2R modulator on the activity of an olfactory CNG channel in cells which co-express an olfactory CNG channel, preferably a human olfactory CNG channel and at least one taste receptor, preferably a human T1R or T2R.

More specifically, it is an object of the invention to provide novel cell-based assays for identifying modulators of a particular T1R or T2R polypeptide or T1R or T2R containing heteromer by detecting changes in intracellular calcium or sodium using cell lines which co-express an olfactory CNG channel and at least one T1R or T2R receptor.

It is another specific object of the invention to provide cell-based assays for identifying T1R and T2R modulators that use techniques which fluorimetrically assay the effect of said putative modulators on intracellular calcium or sodium levels in cell lines that co-express an olfactory CNG channel.

It is a more specific object of the invention to provide an assay for identifying whether a candidate compound is a modulator of a taste receptor comprising:

(i) contacting a test cell that co-expresses (1) at least one functional taste receptor, (2) an olfactory cyclic nucleotide gated channel (oCNGC) subunit and (3) at least one G_{αi/o} protein with a candidate compound;

(ii) detecting whether said candidate compound modulates oCNGC activity based on whether there is a change in intracellular calcium or sodium concentration in said test cell; and

(iii) identifying a candidate compound as a modulator of said functional taste receptor if it results in a detectable change in intracellular calcium or sodium concentration relative to a suitable control cell.

It is another specific object of the invention to provide assays for identifying whether a candidate compound enhances or inhibits the effect of a known T1R or T2R modulatory compound on a T1R or T2R comprising:

(i) contacting a test cell that co-expresses (1) at least one functional T1R or T2R taste receptor, (2) a functional olfactory cyclic nucleotide gated channel (oCNGC) subunit and (3) at least one G_{αi/o} protein with a first compound known to modulate said T1R or T2R taste receptor;

(ii) further contacting an equivalent test cell with the combination of said known T1R or T2R modulator compound and a candidate compound;

(iii) evaluating the effect of said known T1R or T2R modulator compound on oCNG channel function;

(iv) evaluating the effect of the combination of said first known T1R or T2R modulator compound and said candidate compound on oCNG channel function; and

(v) identifying whether the candidate compound is a T1R or T2R modulator based on whether it modulates the effect of said known T1R or T2R modulatory compound on oCNGC activity.

It is another specific object of the invention to provide novel cell lines for identifying compounds that modulate a taste receptor, preferably T1R or T2R, wherein said cell lines co-express (1) at least one functional taste receptor, (2) at least one olfactory cyclic nucleotide gated channel (cCNGC) subunit and (3) at least one G_{αi/o} protein.

It is still another object of the invention to use said T2R or T1R modulatory compounds as flavor-affecting additives, e.g., in foods, beverages and medicaments for human or animal consumption.

It is yet another object of the invention to produce compositions containing T2R or T1R modulatory compounds identified using the subject cell-based assays.

It is a specific object of the invention to provide assays for identifying modulators of T1R or T2R taste receptors wherein at least one T1R to T2R is stably or transiently expressed in a cell, preferably a mammalian cell line such as HEK-293, together with an olfactory CNG channel and a G protein (endogenous or exogenous to the cell) that functionally couples therewith, e.g., a Gα_{i/o}, and the modulator is identified based on its effect on Gα_{i/o} mediated signaling pathways that affect the activity of said olfactory CNG channel, preferably by detecting changes in intracellular calcium or sodium.

### DETAILED DESCRIPTION OF FIGURES

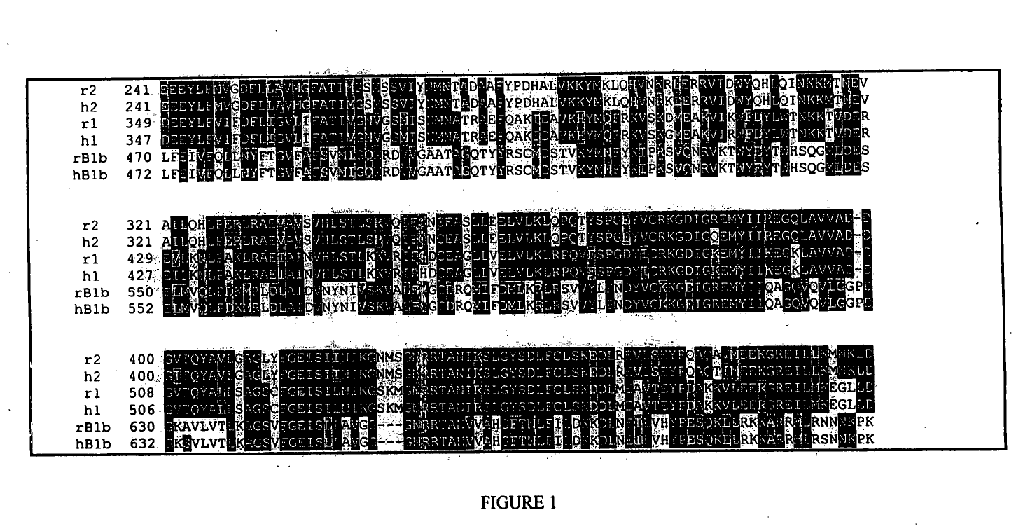

FIG. 1. The human genome contains orthologs of the three rat olfactory CNG channels subunits. Pairwise sequence identities for paralogs range from 30-50%, orthologs 84-90%. Sequences corresponding to the C-terminal cyclic-Nucleotide-binding domains of the rat and human CNG channel subunits are shown. Sequences for the human OCNC1, OCNC2, and OCNCβ1b olfactory CNG channel subunits of this invention are SEQ ID NOs: 1-3, respectively; database sequences for the rat CNG channel subunits OCNC1, OCNC2 and OCNCβ1b are accessions NM₀₁₂₉₂₈, NM₀₅₃₄₉₆, and AJ000515, respectively (SEQ ID Nos: 5-7, respectively).

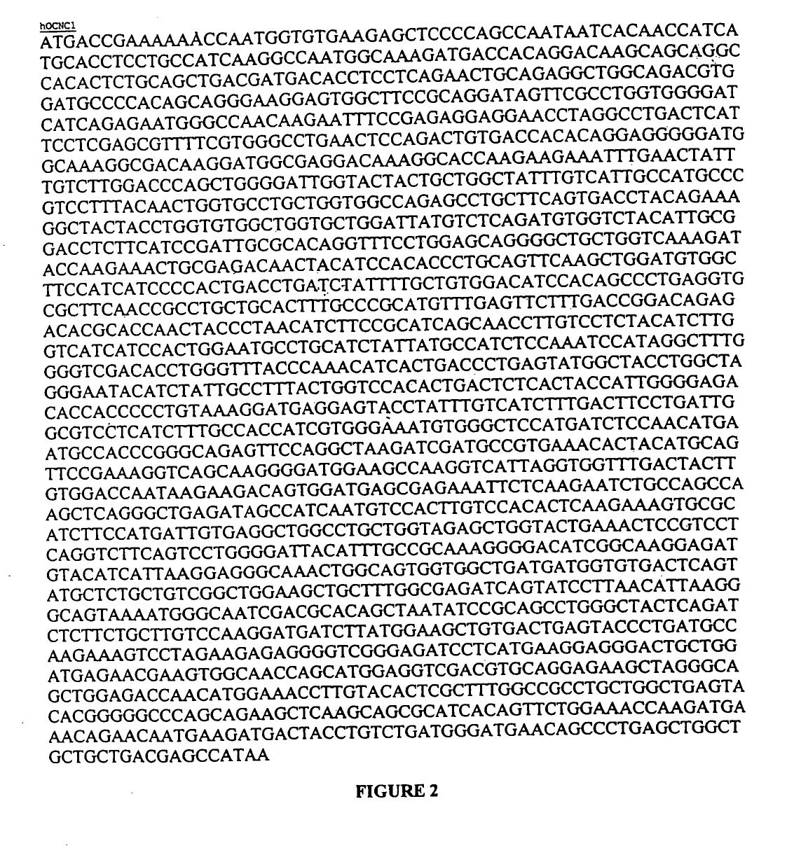

FIG. 2. Sequence of hOCNC1 (SEQ ID NO: 1). This cDNA sequence corresponds to the hOCNC1 gene contained in the cloned genomic interval HSAF002992.

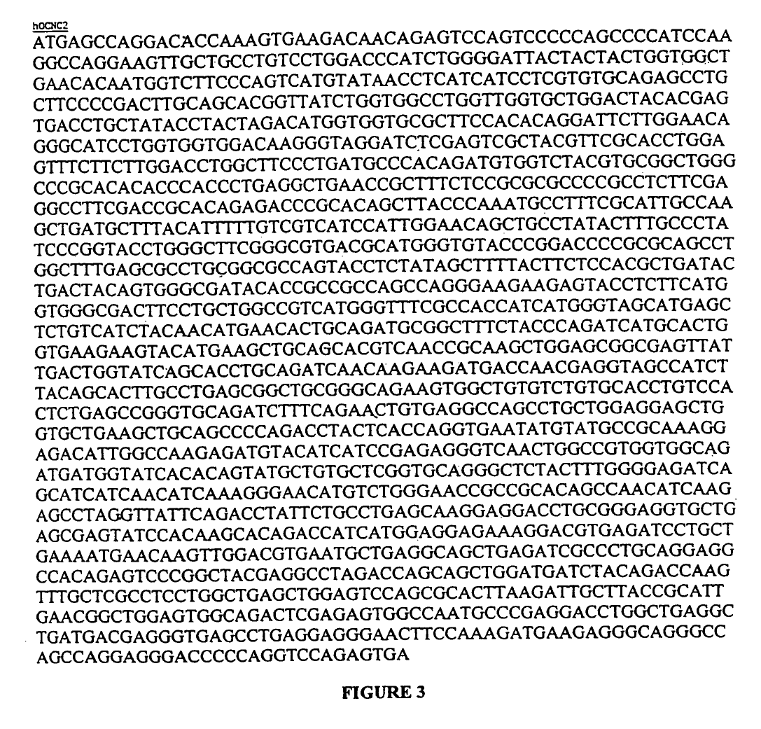

FIG. 3. Sequence of hOCNC2 (SEQ ID NO: 2). This cDNA sequence corresponds to the hOCNC2 gene contained in the cloned genomic interval AC022762.

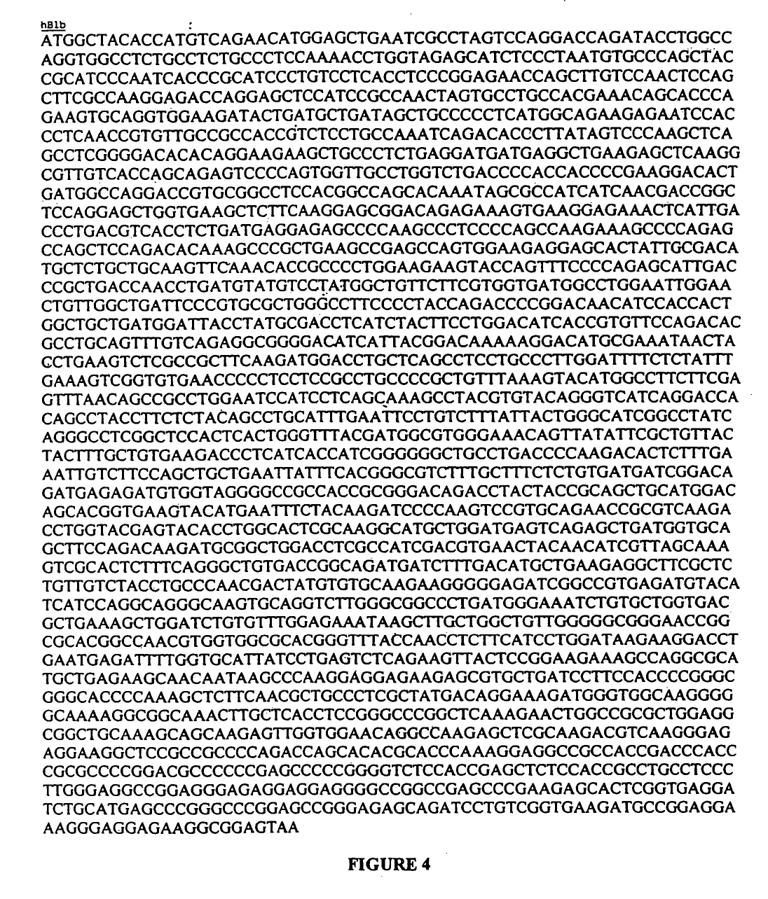

FIG. 4. Sequence of hOCNCβ1b (SEQ ID NO: 3). This cDNA sequence represents a novel allele.

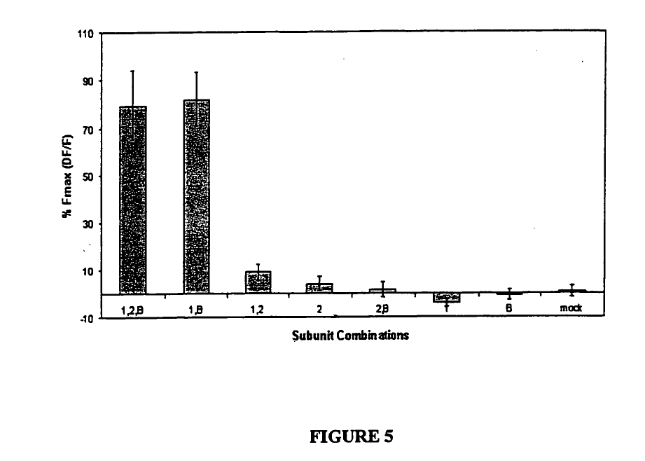

FIG. 5. Olfactory CNG channel activity is dependent on subunit composition. Fluorescence increases at 6 minutes following 50 μM forskolin addition were determined for cells transfected with different combinations of human olfactory CNG channel subunits and loaded with a calcium dye. Activities represent the mean.±.s.e. of 8 independent responses and were normalized to fluorescence increases at 6 minutes following addition of the calcium ionophore ionomycin. OCNC1 is abbreviated as ‘1’, OCNC2 as ‘2’, and OCNCβ1b as ‘B’.

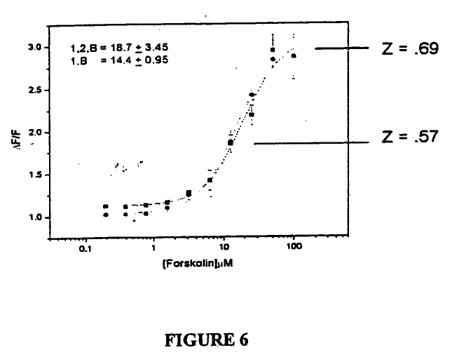

FIG. 6. Membrane-potential-based fluorescent assays for olfactory CNG channel activity are robust. Fluorescence increases at 6 minutes following forskolin addition were determined for cells transfected with different combinations of human olfactory CNG channel subunits and loaded with a membrane-potential dye. Activities represent the mean.±.s.e. of 8 independent responses and were normalized to fluorescence increases at 6 minutes following addition of KCl. EC₅₀ and Z factor values are shown for the two-dose-response curve. OCNC1 is abbreviated as ‘1’, OCNC2 as ‘2’, and OCNCβ1b as ‘B’.

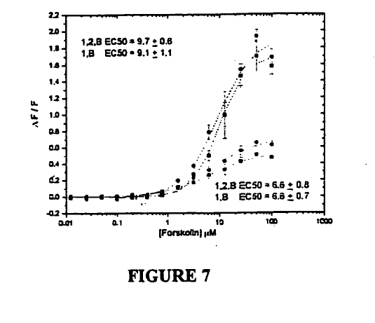

FIG. 7. Calcium-based fluorescent assays for olfactory CNG channel activity are robust. Fluorescence increases at 6 minutes following forskolin addition were determined for cells transfected with different combinations of human ‘channel subunits and loaded with a membrane-potential dye (black) or a calcium dye (grey). Activities represent the mean.±.s.e. of 8 independent responses and were normalize to fluorescence increases at 6 minutes following addition of KCL or ionomycin. EC₅₀ values are shown for the four dose-response curves. OCNC1 is abbreviated as ‘1’, OCNC2 as ‘2’, and β1b as ‘B’.

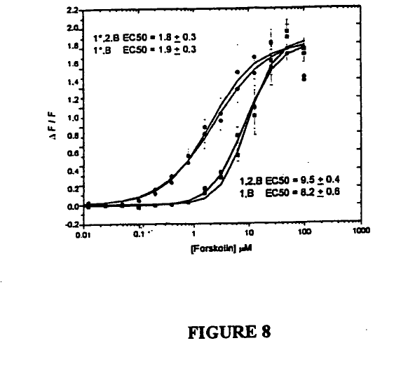

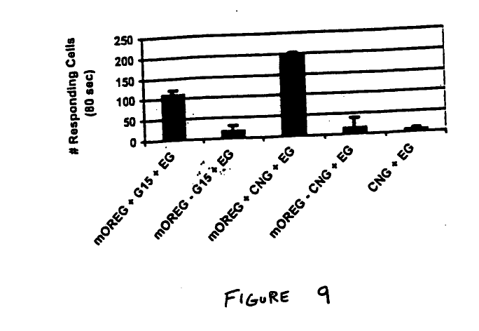

FIG. 8. Human OCNC1 [C458W/E581M] and OCNCβ1b form a sensitized CNG channel. Fluorescence increases at 6 minutes following forskolin addition were determined for cells transfected with different combinations of human olfactory CNG channel subunits and loaded with a membrane-potential dye. Activities represent the mean.±.s.e. of 8 independent responses and were normalized to fluorescence increases at 6 minutes following addition of KCl. EC₅₀ and Z factor values are shown for the two dose-response curves. OCNC1 is abbreviated as ‘1’, OCNC1[C458W/E581M] as ‘1*’, OCNC2 as ‘2’, and OCNCβ1b as ‘B’.

FIG. 9. Olfactor receptor activity can be coupled to the human olfactory CNG channel in heterologous cells. HEK-293 cells were transfected with the mouse olfactory receptor mOREG, OCNC1 [C458W/E581M], OCNC2, and OCNCβ1b, loaded with a calcium dye, and stimulated with the olfactory stimulus eugenol. The number of responding cells was determined by fluorescence miscroscopy and compared to the number of responding HEK-293 cells transfected with G_{α15} and mOREG; [C458W/E581M], OCNC2, and OCNCβ1b.

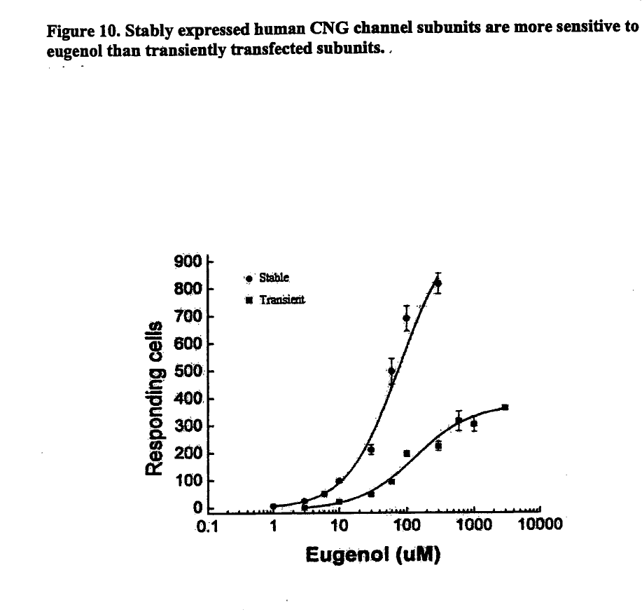

FIG. 10. Stably expressed human CNG channel subunits are more sensitive to eugenol than transiently transfected subunits. HEK-293 cells were stably or transiently expressing the human CNG subunits hOCNC1, hOCNC2 and hOCNCβ1b, and transiently transfected with mOREG. The cells were stimulated with various concentrations of eugenol, and calcium influx was measure using Fluo-4 using fluorescent microscopy. Similar results were obtained with the hOCNC1 [C458W/E581M] and OCNCβ1b subunit (data not shown).

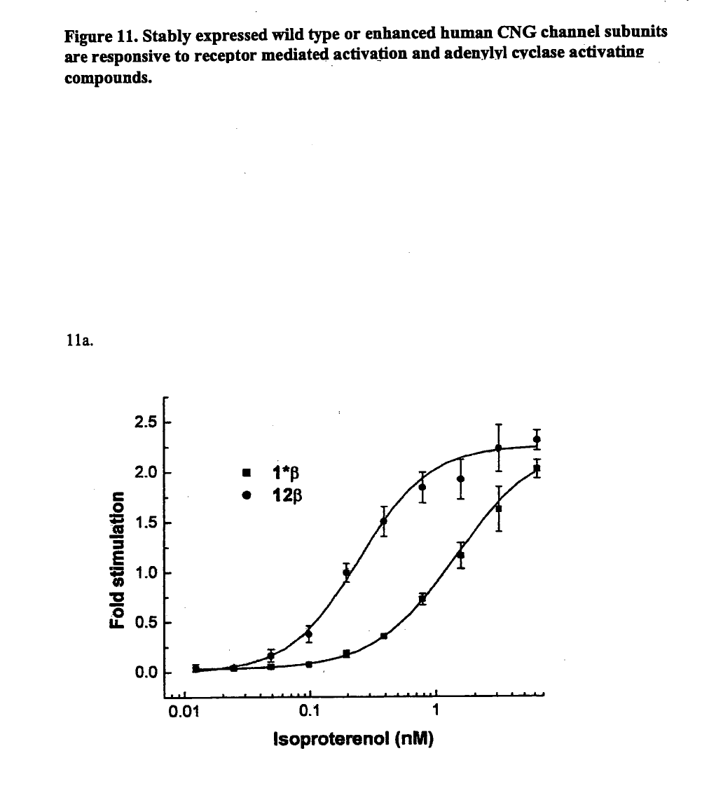

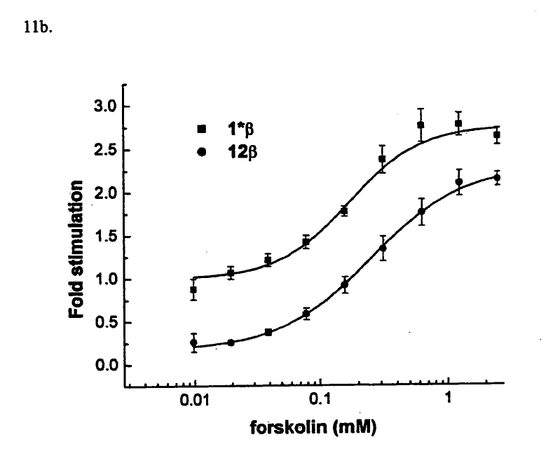

FIG. 11. Stably expressed wild type or enhanced human CNG channel subunits are responsive to receptor mediated activation and adenylyl cyclase activating compounds. In panel (a) of FIG. 11 HEK-293 cells stably expressing eith hOCNC1, hOCNC2 and hOCNCβ1b, or hOCNC1 [C458W/E581M] and OCNCβ1b, were stimulated with various concentrations of the β2 receptor ligand isoproterenol and the calcium influx was measure using Fluo-4 on a FLIPR-1. In panel (b) of FIG. 11 HEK-293 cells stably expressing either hOCNC1, hOCNC2 and hOCNCβ1b, or hOCNC1 [C458W/E581M] and OCNCβ1b, were stimulated with various concentrations of the adenylyl cyclase activator forskolin and the calcium influx was measure using Fluo-4 on a FLIPR-1.

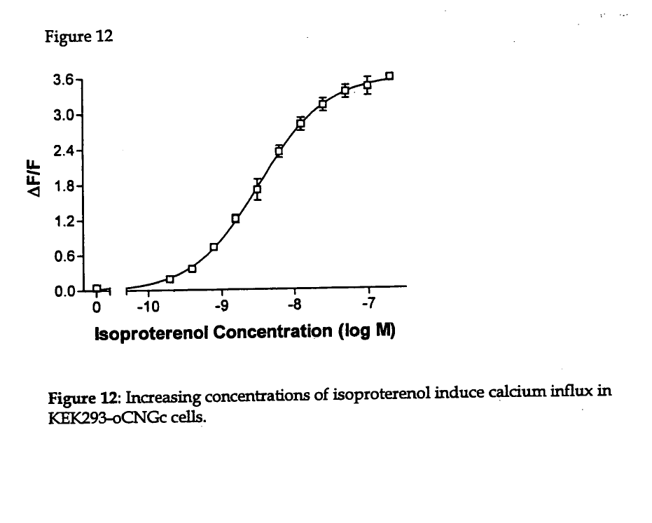

FIG. 12 contains the results of an experiment showing that increasing concentrations of isoproterenol induce calcium influx in HEK 293-oCNGC cells. Delta F/F corresponds to the maximum fluorescence value obtained after stimulation minus the minimum fluorescence measured before stimulation and normalized to the minimum fluorescence measure before stimulation.

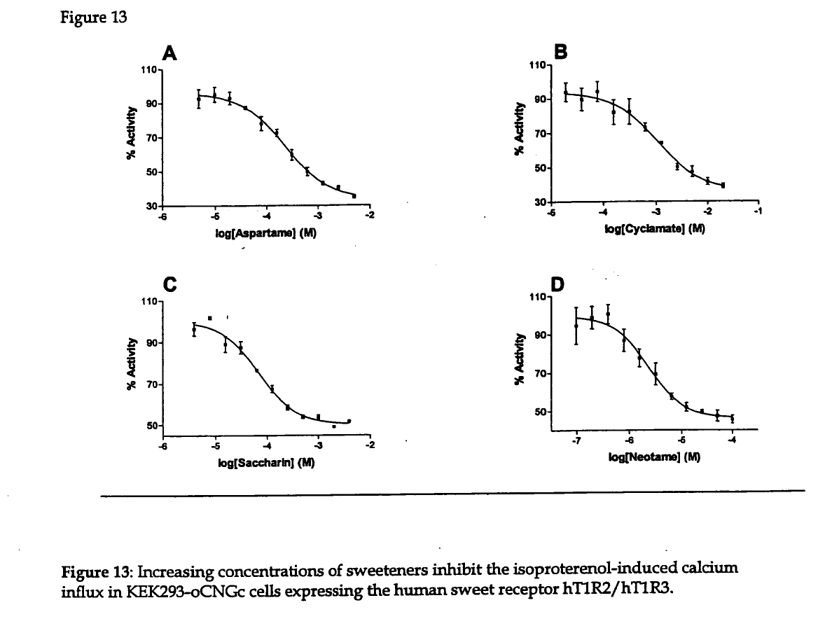

FIG. 13 contains the result of an experiment showing that increasing concentrations of sweeteners inhibit the isoproterenol-induced-calcium influx in HEK 293-oCNGC cells expressing the human sweet receptor hT1R2/hT1R3. Delta F/F values were normalized to the fluorescence obtained after stimulation with 200 nM isoproterenol. Each value corresponds to the mean ± SD of a triplicate determination.

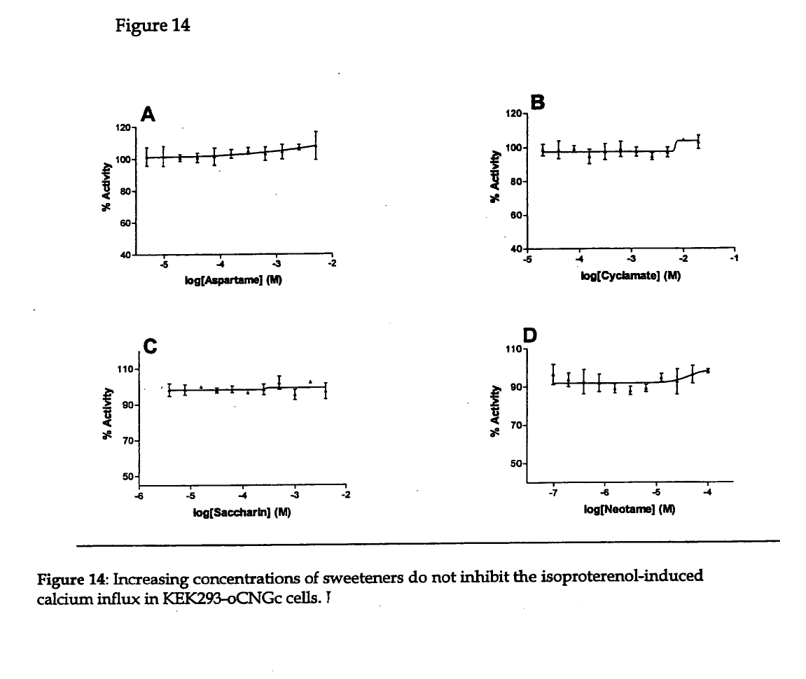

FIG. 14. contains the results of an experiment showing that increasing concentrations of sweeteners do not inhibit the isoproterenol-induced calcium influx in untransfected HEK 293-OCNGC cells. Delta F/F valves were normalized to the fluorescence obtained after stimulation with 200 nM isoproterenol. Each value corresponds to the mean ± SD of a triplicate determination.

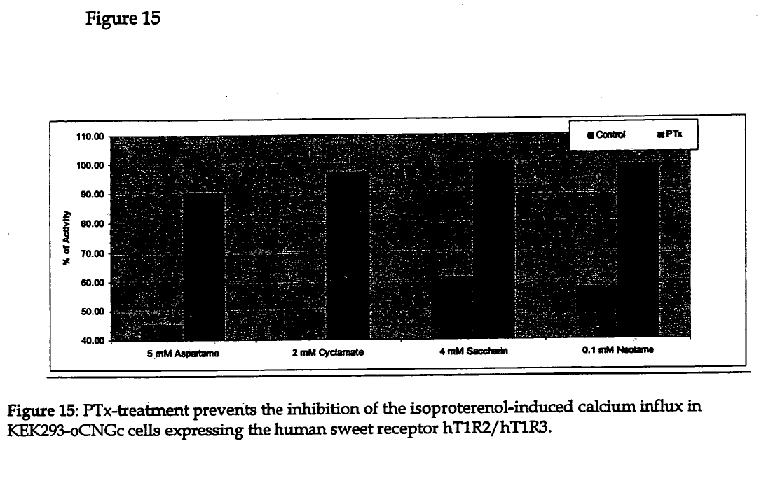

FIG. 15. contains the results of an experiment which reveals that PTX treatment prevents the inhibition of the isoproterenol-induced calcium influx in HEK 293-OcNGC cells expressing the human sweet receptor hT1R2/hT1R3. Cells were left untreated (control) or treated with PTX for 4 hours at 100 ng/ml prior to the experiment. Cells were then stimulated with 200 nM isoproterenol in the presence or absence of the indicated sweeteners (5 mM Aspartame, 2 mM Cyclamate, 4 mM Saccharin and 0.1 mM Neotame).

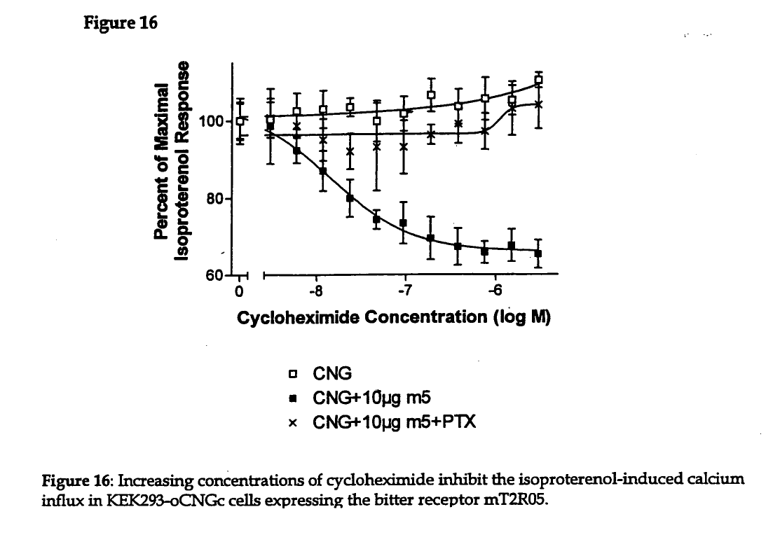

FIG. 16 contains the results of an experiment which reveals that increasing concentrations of cycloheximide inhibit the isoproterenol-induced calcium influx in HEK 293-OCNGC cells expressing the bitter receptor mT2R05. Delta F/F values were normalized to the fluorescence obtained after stimulation with 200 nM isoproterenol. Each value corresponds to the mean +1-SD of a triplicate determination. Cells were also treated with PTX as described for the for the preceding figure. Under these conditions, cycloheximide failed to inhibit the isoproterenol-induced calcium influx. This figure also reveals that un-transfected HEK 293-oCNGC cells did not respond to cycloheximide.

### DETAILED DESCRIPTION OF THE INVENTION

The present invention provides cell-based assays for identifying compounds that modulate the activity of specific taste receptors, T1R or T2R taste receptors or which modulate the effect of another T1R or T2R modulator compound, e.g., a sweetener, umami compound or bitter compound preferably by assaying their effect on the activity of an olfactory CNG channel contained in a cell line that co-expresses the olfactory CNG channel, at least one functional T1R or T2R, and a G protein that functionally couples therewith. (As defined herein, “modulators” according to the invention include T1R or T2R agonists, antagonists and enhancers.)

These cell-based assays are an extension of two of Applicants' prior discoveries, the first being that T1Rs and T2Rs functionally couple to G proteins other than cc-gustducin or Gα₁₅, particularly Gα_{i/o} proteins such as Gαᵢ. As discussed in detail in Applicants' earlier application, U.S. Ser. No. 10/770,127 filed Feb. 3, 2004 incorporated by reference herein it has been shown that bitter compounds such as cycloheximide specifically activate ERK1/2 mitogen activated kinases in cells expressing a T2R and Gαᵢ and also that cycloheximide inhibits forskolin-induced cAMP accumulation. Further, it has been shown that natural and artificial sweetener compounds activate ERK1/2 in cells expressing hT1R2/hT2R3 and a Gα_{i/o} protein and that monosodium glutamate specifically activates ERK1/2 in cells expressing hT1R1/hT1R3 and Gαᵢ protein and further completely inhibits forskolin-induced cAMP accumulation in such cells; and that activation of ERK1/2 by these compounds is totally abolished by treatment with pertussis toxin. These results provide compelling evidence that the T1R and T2R receptors indeed couple and activate ERK1/2 and inhibit adenylyl cyclase through Gα_{i/o}.

Secondly, this invention pertains to Applicants'previous discovery relating to isolated nucleic acid sequences that encode human olfactory cyclic nucleotide gated (CNG) channel subunits, and the corresponding polypeptides, and mammalian cell-based high throughput assays which identify compounds that modulate the human olfactory CNG channel. These assays include fluorescence-based assays which screen for compounds that result in detectable change in intracellular cation levels, e.g., calcium or sodium, wherein these changes are detected fluorimetrically using cation sensitive dyes. These discoveries form the basis of U.S. Ser. No. 10/189,507 filed Jul. 8, 2002 incorporated by reference in its entirety herein.

Specifically, the present invention relates to the discovery that the activity of taste receptors, particularly T1R receptors (umami and sweet) and T2Rs, (bitter taste receptors) can be indirectly detected using techniques that monitor the activity of an olfactory CNG channel using cell lines that co-express such olfactory CNG channel and such taste receptors. As described supra it is known that T1Rs, and T2Rs belong to the family of G protein-coupled receptor (GPCRs) characterized by 7 transmembrane domains which function predominantly through activation of specific G proteins that in turn activate specific effective enzymes, such as phospholipase C (PLC) inside the cell.

It is also known that an olfactory CNG channel is activated by an increase in the concentration of intracellular messenger cAMP. Upon activation by cAMP, olfactory CNG channels become selectively permeable to extracellular ions, e.g., calcium and sodium resulting in an increase in intracellular calcium and sodium concentrations.

Surprisingly, the present inventors have discovered that the activation of taste receptors, i.e., T1Rs or T2Rs which are members of the GPCR family can inhibit olfactory CNG channel activity, e.g., as evidenced by a detectable decrease in ion influx through the olfactory CNG channel, e.g., calcium or sodium. This discovery correlates with Applicants' previous discovery that T1Rs and T2Rs are capable of functionally coupling with G_{αi/o} proteins and thereby activating G_{αi} signaling pathways that affect downstream effectors such as cAMP, MAPK, adenylyl cyclase, among others. The experimental results provided herein suggest that the inhibition of oCNGC activity by activation of T1Rs and T2Rs is dependent on the functional coupling of T1Rs and T2Rs to G_{αi/o} proteins resulting in a decrease in cAMP which in turn results in a decrease in levels of intracellular calcium or sodium flowing through the CNG channel. Therefore, the invention in its preferred embodiment, provides cell-based assays for indirectly determining the effect of a candidate compound on T1R or T2R activity based on the effect of such compound on the activity of an olfactory CNG channel which is comprised in a cell line that expresses such olfactory CNG channel in association with at least one T1R or T2R receptor polypeptide.

More specifically, the invention provides fluorimetric cell-based assays and materials for use therein that provide for the rapid and accurate identification of taste modulatory compounds. These taste modulatory compounds have potential utility as flavor enhancers or flavor additives for incorporation in foods and beverages for human or animal consumption.

### DEFINITIONS AND ABBREVIATIONS

Prior to providing a detailed description of the invention, and its preferred embodiments, the following definitions and abbreviations are provided. Otherwise all terms have their ordinary meaning as they would be construed by one skilled in the relevant art.

### ABBREVIATIONS USED

Some abbreviations used in this application are set forth below.

cAMP: 3′ 5′-cyclic adenosine monophsphate, TRCs: Taste receptor cells, GPCRs: G protein-coupled receptors, MSG: Monosodium glutamate, PDE: phosphodiesterase; MAPK: Mitogen activated protein kinase, IMP: inosine monophosphate, PTX: pertussis toxin, EGF: Epidermal growth factor, PKC: Protein kinase C, RTKs: Receptor tyrosine kinases, PKA: Protein kinase A, ACs: Adenylyl cyclases, cNMP: cyclic nucleotide monophosphate, CREB: cAMP response element-binding protein, PLCP2: Phospholipase CP2, Trp: Transient receptor potential.

“Taste cells” include neuroepithelial cells that are organized into groups to form taste buds of the tongue, e.g., foliate, fungiform, and circumvallate cells (see, e.g., Roper et al., Ann. Rev. Neurosci. 12:329-353 (1989)). Taste cells are also found in the palate and other tissues, such as the esophagus and the stomach.

“T1R” refers to one or more members of a family of G protein-coupled receptors that are expressed in taste cells such as foliate, fungiform, and circumvallate cells, as well as cells of the palate, and esophagus (see, e.g., Hoon et al., Cell, 96:541-551 (1999), herein incorporated by reference in its entirety). The definition of “T1R” should further be construed based on DNA and amino acid sequences disclosed in the Senomyx and University of California patent applications and publications incorporated by reference herein. Members of this family are also referred to as GPCR-B3 and TR1 in WO 00/06592 as well as GPCR-B4 and TR2 in WO 00/06593. GPCR-B3 is also herein referred to as rT1R1, and GPCR-B4 is referred to as rT1R2. Taste receptor cells can also be identified on the basis of morphology, or by the expression of proteins specifically expressed in taste cells. T1R family members may have the ability to act as receptors for sweet or umami taste transduction, or to distinguish between various other taste modalities. T1R sequences, including hT1R1, hT1R2 and hT1R3 are identified in the Senomyx and University of California patent applications incorporated by reference in their entirety herein and are provided infra, in an Appendix after the claims.

“T1R” nucleic acids encode a family of GPCRs with seven transmembrane regions that have “G protein-coupled receptor activity,” e.g., they may bind to G proteins in response to extracellular stimuli and promote production of second messengers such as IP3, cAMP, cGMP, and Ca²⁺ via stimulation of enzymes such as phospholipase C and adenylate cyclase (for a description of the structure and function of GPCRs, see, e.g., Fong, TM Cells Signal. 8(3):217-224 (1996) and Baldwin, et al., J. Mol. Biol. 272(1):144-164 (1997). A single taste cell may contain many distinct T1R polypeptides.

The term “T1R” family therefore refers to polymorphic variants, alleles, mutants, and interspecies homologus that: (1) have at least about 35 to 50% amino acid sequence identity, optionally about 60, 75, 80, 85, 90, 95, 96, 97, 98, or 99% amino acid sequence identity to a T1R polypeptide, preferably those identified in the patent applications incorporated by reference herein, over a window of about 25 amino acids, optionally 50-100 amino acids; (2) specifically bind to antibodies raised against an immunogen comprising an amino acid sequence preferably selected from the group consisting of the T1R polypeptide sequence disclosed in the patent applications incorporated by reference herein and conservatively modified variants thereof; (3) are encoded by a nucleic acid molecule which specifically hybridize (with a size of at least about 100, optionally at least about 500-1000 nucleotides) under stringent hybridization conditions to a sequence selected from the group consisting of the T1R nucleic acid sequences contained in the applications incorporated by reference in their entirety herein, and conservatively modified variants thereof; or (4) comprise a sequence at least about 35 to 50% identical to an amino acid sequence selected from the group consisting of the T1R amino acid sequence identified in the patent applications incorporated by reference in their entirety herein.

The term “T2R” refers to one or more members of a family of G protein coupled receptors that are expressed in taste cells, specifically, the tongue and palate epithelia. In particular, T2R includes the particular genes identified in the Senomyx and University of California applications relating to T2Rs incorporated by reference in their entirety herein. T2Rs are genetically linked to loci associated with bitter taste perception in mice and humans. More specifically, the term “T2R” and terms including T2R, e.g., T2R04 or T2R05 refers generally to isolated T2R nucleic acids, isolated polypeptides encoded by T2R nucleic acids, and activities thereof T2R nucleic acids and polypeptides can be derived from any organism. The terms “T2R” and terms including “T2R” also refer to polypeptides comprising receptors that are activated by bitter compounds, and to nucleic acids encoding the same. Thus both T1Rs and T2Rs comprise different families of chemosensory GPCRs. Sequences of various T2Rs are also contained in the Appendix that precedes the claims.

“Functional Human Olfactory CNG Channel” or “human oCNGC” refers to an olfactory neuron-specific CNG unit comprises of at least one olfactory CNG channel subunit, preferably a human olfactory CNG channel subunit, variant or fragment thereof. Such CNG subunits include OCNC1, OCNC2 and OCNCβb1. A functional channel will be sufficiently permeable to extracellular cations, particularly sodium or calcium, to produce detectable changes in intracellular cations, e.g., sodium or calcium, using a membrane potential-sensitive fluorescent dye or a calcium-sensitive fluorescent dye.

“Human Olfactory CNG Channel Subunit” refers to a human ortholog of a rat olfactory polypeptide selected from rat OCNC1, OCNC2 and OCNCβ1b or a nucleic acid sequence that exhibits at least 60%, preferably at least 70%, more preferably 80-90%, and still more preferably at least 90-99% of sequence identity with OCNC1 and human OCNCβ1b sequences disclosed in U.S. Ser. No. 10/189,507 and contained in the Appendix of sequences that precedes the claims infra. Further variants or fragments can be selected based on their ability upon expression alone or in combination with other CNG units to produce functional olfactory (calcium permeable CNG subunits).

In a preferred embodiment, the human ortholog will comprise one of these sequences, or will comprise a fragment or variant thereof that exhibits at least 80%, more preferably at least 90%, and still more preferably at least 95-99% identical thereto and/or sequences which specifically hybridize thereto according to one of the various stringent hybridization conditions defmed infra. In a particularly preferred embodiment the human ortholog will comprise one or more mutations that enhance the sensitivity of the human orthology to cAMP. Exemplary mutuations are described infra.

G proteins are heterotrimeric proteins composed of a single α subunit complexed with the βγ dimer. Molecular cloning has resulted in the identification of eligible distinct α. subunits, five β subunits, and 12 γ subunits. G proteins are usually divided into four subfamilies Gᵢ, Gₛ, G_{q}, and G₁₂ based on the sequence similarity of the Gα subunit. Several lines of evidence suggest that the interaction between a given GPCR and its cognate G protein involves multiple sites of contact on both proteins. All three intracellular loops as well as the carboxyl terminal tail of the receptor have been implicated. The GPCR is thought to interact with all three subunits of the G protein. As the receptor-G protein interaction can be disrupted by a number of treatments that block the carboxyl terminus, including pertussis toxin-catalyzed ADP-ribosylation of G_{α} and binding of monoclonal antibodies, the carboxy terminal region of the Gα subunit has been the most intensely investigated contact site. These studies have shown that the G_{α}. carboxy-terminal region is important not only to the interaction, but also plays a critical role in defining receptor specificity (Hamm et al., Science 241: 832-5 (1988); Osawa et al., J. Biol. Chem. 270: 31052-8 (1995); Garcia et al., EMBO 14: 4460-9 (1995); Sullivan et al., Nature 330: 758-760 (1987); Rasenick et al., J. Biol. Chem. 269: 21519-21525 (1994); West et al., J. Biol. Chem. 260: 14428-30 (1985); Conklin et al., 1993, Nature 363: 274-276; Conklin et al., Mol. Pharmacol. 50: 885-890 (1996)). Furthermore, it has been shown that peptides corresponding to the carboxy terminal region of a G_{αi} subunit can block GPCR signaling events (Hamm et al., Science 241: 832-5 (1988); Gilchrist et al., J. Biol. Chem 273: 14912-19 (1998)). However, prior to the Applicants' earlier invention disclosed in U.S. Ser. No. 10/770,127, it was unknown that Gᵢ proteins were capable of functionally coupling to T1Rs and T2Rs.

Topologically, certain chemosensory GPCRs have an “N-terminal domain;” “extracellular domains;” “transmembrane domains” comprising seven transmembrane regions, and corresponding cytoplasmic, and extracellular loops; “cytoplasmic domains,” and a “C-terminal domain” (see, e.g., Hoon et al., Cell, 96:541-551 (1999); Buck & Axel, Cell, 65:175-187 (1991)). These domains can be structurally identified using methods known to those of skill in the art, such as sequence analysis programs that identify hydrophobic and hydrophilic domains (see, e.g., Stryer, Biochemistry, (3rd ed. 1988); see also any of a number of Internet-based sequence analysis programs. Such domains are useful for making chimeric proteins and for in vitro assays of the invention, e.g., ligand binding assays.

“Extracellular domains” therefore refers to the domains of T1R and T2R polypeptides that protrude from the cellular membrane and are exposed to the extracellular face of the cell. Such domains generally include the “N terminal domain” that is exposed to the extracellular face of the cell, and optionally can include portions of the extracellular loops of the transmembrane domain that are exposed to the extracellular face of the cell, i.e., the loops between transmembrane regions 2 and 3, between transmembrane regions 4 and 5, and between transmembrane regions 6 and 7.

The “N-terminal domain” region starts at the N-terminus and extends to a region close to the start of the first transmembrane domain. More particularly, in one embodiment of the invention, this domain starts at the N-terminus and ends approximately at the conserved glutamic acid at amino acid position 563 plus or minus approximately 20 amino acids. These extracellular domains are useful for in vitro ligand-binding assays, both soluble and solid phase. In addition, transmembrane regions, described below, can also bind ligand either in combination with the extracellular domain, and are therefore also useful for in vitro ligand-binding assays.

Transmembrane domain,” which comprises the seven “transmembrane regions,” refers to the domain of T1R or T2R polypeptides that lies within the plasma membrane, and may also include the corresponding cytoplasmic (intracellular) and extracellular loops. In one embodiment, this region corresponds to the domain of T1R or T2R family members. In the case of T1R family member this starts approximately at the conserved glutamic acid residue at amino acid position 563 plus or minus 20 amino acids and ends approximately at the conserved tyrosine amino acid residue at position 812 plus or minus approximately 10 amino acids. The seven transmembrane regions and extracellular and cytoplasmic loops can be identified using standard methods, as described in Kyte & Doolittle, J. Mol. Biol., 157:105-32 (1982)), or in Stryer, supra.

“Cytoplasmic domains” refers to the domains of T1R or T2R polypeptides that face the inside of the cell, e.g., the “C-terminal domain” and the intracellular loops of the transmembrane domain, e.g., the intracellular loop between transmembrane regions 1 and 2, the intracellular loop between transmembrane regions 3 and 4, and the intracellular loop between transmembrane regions 5 and 6. “C-terminal domain” refers to the region that spans the end of the last transmembrane domain and the 0-terminus of the protein, and which is normally located within the cytoplasm. In one embodiment, this region starts at the conserved tyrosine amino acid residue at position 812 plus or minus approximately 10 amino acids and continues to the C-terminus of the polypeptide.

The term “ligand-binding region” or “ligand-binding domain” refers to sequences derived from a taste receptor, particularly a taste receptor that substantially incorporates at least the extracellular domain of the receptor. In one embodiment, the extracellular domain of the ligand-binding region may include the N-terminal domain and, optionally, portions of the transmembrane domain, such as the extracellular loops of the transmembrane domain. The ligand-binding region may be capable of binding a ligand, and more particularly, a compound that enhances, mimics, blocks, and/or modulates taste, e.g., sweet, bitter, or umami taste. In the case of T2Rs, the compound bound by the ligand binding region will modulate bitter taste. In the case of T1Rs, the compound bound by the ligand-binding region will modulate sweet or umami taste.

The phrase “heteromultimer” or “heteromultimeric complex” in the context of the T1R receptors or polypeptides used in the assays of the present invention refers to a functional association of at least one T1R receptor and another receptor, typically another T1R receptor polypeptide (or, alternatively another non-T1R receptor polypeptide). For clarity, the functional co-dependence of the T1Rs is described in this application as reflecting their possible function as heterodimeric taste receptor complexes. However, as discussed in Senomyx patent applications and publications, which are incorporated by reference herein, functional, co-dependence may alternatively reflect an indirect interaction. For example, T1R3 may function solely to facilitate surface expression of T1R1 and T1R2 which may act independently as taste receptors. Alternatively, a functional taste receptor may be comprised solely of T1R3 which is differentially processed under the control of T1R1 or T1R2, analogous to RAMP-dependent processing of the calcium-related receptor. By contrast, in the case of T2Rs the eukaryotic cells used in the subject MAPK assays will preferably express a single T2R.

The phrase “modulator” or “modulatory compound” means any compound that itself affects the activity of a T1R or T2R or modulates (affects) the effect of another compound on T1R or T2R activity. Herein, modulation is preferably determined indirectly using cell-based assays that detect the effect of a putative modulator on Gi signaling pathways, e.g., assays that detect the effect of a compound on an olfactory CNG channel in a cell line that co-expresses the olfactory CNG channel, a T1R or T2R and a G_{αi/o} protein.

The phrase “functional effects” in the context of assays for testing compounds that modulate at least one T1R or T2R family member mediated taste transduction includes the determination of any parameter that is indirectly or directly under the influence of the receptor, e.g., functional, physical and chemical effects. It includes ligand binding, changes in ion flux, membrane potential, current flow, transcription, G protein binding, GPCR phosphorylation or dephosphorylation, conformation change-based assays, signal transduction, receptor-ligand interactions, second messenger concentrations (e.g., cAMP, cGMP, IP3, or intracellular Na⁺ or Ca²⁺), in vitro, in vivo, and ex vivo and also includes other physiologic effects such increases or decreases of neurotransmitter or hormone release. In the present invention, the assays will generally measure the effect of a compound on the activity of an olfactory CNG channel in a cell line that co-expresses the CNG channel and at least one functional T1R or T2R using means for detecting olfactory CNG activity that are known in the art, e.g., disclosed in Applicants' earlier patent application, U.S. Ser. No. 10/189,507, incorporated by reference in its entirety herein. In the present invention, the effect of a putative modulator of a T1R or T2R will be determined indirectly based on its effect on the activity of a olfactory CNG channel, preferably by detecting changes in intracellular calcium or sodium, e.g., using flourimetric assay techniques.

By “determining the functional effect” in the context of assays is meant assays for a compound that increases or decreases a parameter that is indirectly or directly under the influence of at least one T1R or T2R family member, e.g., functional, physical and chemical effects. Such functional effects can be measured by any means known to those skilled in the art, e.g., changes in spectroscopic characteristics (e.g., fluorescence, absorbency, refractive index), hydrodynamic (e.g., shape), chromatographic, or solubility properties, patch clamping, voltage-sensitive dyes, whole cell currents, radioisotope effilux, inducible markers, oocyte T1R or T2R gene expression; tissue culture cell T1R or T2R expression; transcriptional activation of T1R or T2R genes; ligand-binding assays; voltage, membrane potential and conductance changes; ion flux assays; changes in intracellular second messengers such as cAMP, cGMP, and inositol triphosphate (IP3); changes in intracellular calcium or sodium levels; neurotransmitter release, conformational assays and the like. In the present invention, the effect of a putative modulator compound will be preferably assayed based on its effect the activity of a human olfactory CNG channel.

“Mmodulators” of T1R or T2R genes or proteins are used to refer to inhibitory, activating, or modulating molecules identified using in vitro and in vivo assays that directly or indirectly identify compounds that affect taste transduction, e.g., ligands, agonists, antagonists, inverse agonists, and their homologues and mimetics. These compounds themselves modulate T1R or T2R activity or modulate the effect of another compound on T1R or T2R activity. Therefore, T1R or T2R modulators according to the invention expressly include T1R or T2R antagonists, agonists and enhancers. In the present invention, these molecules will preferably be identified using the subject cell-based olfactory CNG channel assays. In preferred embodiments, the “inhibitors” will block taste of a known bitter compound and “enhancers” will enhance the taste of another sweet or umami compound or compounds.

Inhibitors are compounds that, e.g., bind to, partially or totally block stimulation, decrease, prevent, delay activation, inactivate, desensitize, or down regulate the activity of a receptor, e.g., a T1R or T2R and taste transduction, e.g., antagonists. In the preferred methods, such inhibitors will enhance olfactory CNG channel activity. Activators are compounds that, e.g., bind to, stimulate, increase, open, activate, facilitate, enhance activation, sensitize, or up regulate the activity of a receptor, e.g., T1R or T2R olfactory receptor or a CNG channel, e.g., agonists. In the preferred methods, T1R or T2R activities will inhibit olfactory CNG channel activity. Modulators include compounds that, e.g., alter the interaction of a receptor with: extracellular proteins that bind activators or inhibitor (e.g., ebnerin and other members of the hydrophobic carrier family); G proteins; kinases (e.g., homologues of rhodopsin kinase and beta adrenergic receptor kinases that are involved in deactivation and desensitization of a receptor); and arresting, which also deactivate and desensitize receptors. Modulators can include genetically modified versions of T1R or T2R family members, e.g., with altered activity, as well as naturally occurring and synthetic ligands, antagonists, agonists, small chemical molecules and the like. Such assays for inhibitors and activators include, e.g., expressing T1R or T2R family members in association with an olfactory CNG channel cells or cell membranes, applying putative modulator compounds, in the presence or absence of tastants, e.g., sweet, umami or bitter tastants, and then determining the functional effects on taste transduction, as described above. Samples or assays comprising T1R or T2R family members and an olfactory CNG channel that are treated with a potential activator, inhibitor, or modulator are compared to control samples without the inhibitor, activator, or modulator to examine the extent of modulation. Positive control samples (e.g., a sweet, umami, or bitter tastant without added modulators) are assigned a relative activity value of 100%. In the present invention, such inhibitors are identified indirectly based on the effect of a putative inhibitor on olfactory CNG channel activity.

Negative control samples (e.g., buffer without an added taste stimulus) are assigned a relative T1R or T2R activity value of 0%. Inhibition of a T1R or T2R or oCNGC is achieved when a mixture of the positive control sample and a modulator result in the T1R or T2R or oCNGC activity value relative to the positive control is about 80%, optionally 50% or 25-0%. Activation of a T1R or T2R or oCNGC by a modulator alone is achieved when the activity value relative to the positive control sample is 10%, 25%, 50%, 75%, optionally 100%, optionally 150%, optionally 200-500%, or 1000-3000% higher.

The terms “purified,” “substantially purified,” and “isolated” as used herein refer to the state of being free of other, dissimilar compounds with which the compound of the invention is normally associated in its natural state, so that the “purified,” “substantially purified,” and “isolated” subject comprises at least 0.5%, 1%, 5%, 10%, or 20%, and most preferably at least 50% or 75% of the mass, by weight, of a given sample. In one preferred embodiment, these terms refer to the compound of the invention comprising at least 95% of the mass, by weight, of a given sample. As used herein, the terms “purified,” “substantially purified,” and “isolated,” when referring to a nucleic acid or protein, also refers to a state of purification or concentration different than that which occurs naturally in the mammalian, especially human body. Any degree of purification or concentration greater than that which occurs naturally in the mammalian, especially human, body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones (see e.g., Oligonucleotides and Analogues, a Practical Approach, ed. F. Eckstein, Oxford Univ. Press (1991); Antisense Strategies, Annals of the N. Y. Academy of Sciences, Vol. 600, Eds. Baserga et al. (NYAS 1992); Milligan J. Med. Chem. 36:1923-1937 (1993); Antisense Research and Applications (1993, CRC Press), Mata, Toxicol. Appl. Pharmacol. 144:189-197 (1997); Strauss-Soukup, Biochemistry 36:8692-8698 (1997); Samstag, Antisense Nucleic Acid Drug Dev, 6:153-156 (1996)).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues (Batzer et al, Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “plasma membrane translocation domain” or simply “translocation domain” means a polypeptide domain that, when incorporated into a polypeptide coding sequence, can with greater efficiency “chaperone” or “translocate” the hybrid (“fusion”) protein to the cell plasma membrane than without the domain. For instance, a “translocation domain” may be derived from the amino terminus of the bovine rhodopsin receptor polypeptide, a 7-transmembrane receptor. However, rhodopsin from any mammal may be used, as can other translocation facilitating sequences. Thus, the translocation domain is particularly efficient in translocating 7-transmembrane fusion proteins to the plasma membrane, and a protein (e.g., a taste receptor polypeptide) comprising an amino terminal translocating domain will be transported to the plasma membrane more efficiently than without the domain. However, if the N-terminal domain of the polypeptide is active in binding, as with the T1R or T2R receptors of the present invention, the use of other translocation domains may be preferred.

The “translocation domain,” “ligand-binding domain”, and chimeric receptors compositions described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide, which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide, is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gin; ile/leu or val; leu/ile or val; lys/arg or gln or glu; met/leu or tyr or lie; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides, e.g., translocation domains, ligand-binding domains, or chimeric receptors of the invention. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH2-NH), ethylene, olefin (CH═CH), ether (CH₂—O), thioether (CH2-S), tetrazole (CN₄), thiazole, retroamide, thioamide, or ester (see, e.g., Spatola, Chemistry and Biochemistry of Amino Acids, Peptides and Proteins, Vol. 7, pp 267-357, “Peptide Backbone Modifications,” Marcell Dekker, NY (1983)). A polypeptide can also be characterized as a mimetic by containing all or some non-natural residues in place of naturally occurring amino acid residues; non-natural residues are well described in the scientific and patent literature.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions.

An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

As used herein, a “stable cell line” refers to a cell line, which stably, i.e. over a prolonged period, expresses a heterologous nucleic sequence, i.e., a T1R, T2R, olfactory CNG channel or G protein. In preferred embodiments, such stable cell lines will be produced by transfecting appropriate cells, typically mammalian cells, e.g., HEK-293 cells, with a linearized vector that contains a T1R or T2R expression construct that expresses at least one T1R or T2R, i.e., T1R1, T1R2 and/or T1R3 or a T2R and with a linearized vector comprising human olfactory CNG channel subunit nucleic acid sequences. Most preferably, stable cell lines that express a functional T1R or T2R receptor will be produced by co-transfecting two linearized plasmids that express hT1R1 and hT1R3 or hT1R2 and hT1R3 or a single linearized plasmid that expresses a specific T2R and a linearized plasmid containing olfactory CNG channel subunit sequences, which optionally may be mutated, and an appropriate selection procedure to generate cell lines having these genes stably integrated therein. Most preferably, the cell line will also stably express a G protein preferably a G_{αi/o} such as G_{αi} or G_{α15}.

“Antibody” refers to a polypeptide comprising a framework region from an immunoglobulin gene or fragment thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable light chain” (VL) and “variable heavy chain” (VH) refer to these light and heavy chains respectively.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-T1R” antibody is an antibody or antibody fragment that specifically binds a polypeptide encoded by a T1R gene, cDNA, or a subsequence or variant thereof.

An “anti-T2R” antibody is an antibody or antibody fragment that specifically binds a polypeptide encoded by T2R gene, cDNA, or a subsequence or variant thereof.

A “ligand that detects cAMP” is any moiety that specifically detects cAMP levels.

A “ligand that detects intracellular calcium or sodium” is any moiety that specifically detects Ca⁺⁺ or Na⁺ levels.

The term “immunoassay” is an assay that uses an antibody to specifically bind an antigen. The immunoassay is characterized by the use of specific binding properties of a particular antibody to isolate, target, and/or quantify the antigen. In a preferred embodiment of the invention, MAPK activity or cAMP levels will be immunoassayed in eukaryotic cells using an antibody that specifically recognizes an activated form of MAPK or cAMP.

The phrase “specifically (or selectively) binds” to an antibody or, “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a T1R or T2R family member from specific species such as rat, mouse, or human can be selected to obtain only those polyclonal antibodies that are specifically immunoreactive with the T1R or T2R polypeptide or an immunogenic portion thereof and not with other proteins, except for orthologs or polymorphic variants and alleles of the T1R or T2R polypeptide. This selection may be achieved by subtracting out antibodies that cross-react with T1R or T2R molecules from other species or other T1R or T2R molecules. Antibodies can also be selected that recognize only T1R GPCR family members but not GPCRs from other families. In the case of antibodies to activated MAPKs, suitable polyclonal and monoclonal antibodies are commercially available.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual, (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

The term “expression vector” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes which contain only the minimum elements needed for transcription of the recombinant nucleic acid.

By “host cell” is meant a cell that contains an expression vector and supports the replication or expression of the expression vector. Host cells may be prokaryotic cells such as E. coli, or eukaryotic cells such as yeast, insect, amphibian, worm or mammalian cells such as CHO, Hela, BHK, HEK, HEK-293T, COS, NIH3T3, SWISS3T3, HEK-293, and the like, e.g., cultured cells, explants, and cells in vivo.

The terms “a,” “an,” and “the” are used in accordance with long-standing convention to refer to one or more.

The term “about”, as used herein when referring to a measurable value such as a percentage of sequence identity (e.g., when comparing nucleotide and amino acid sequences as described herein below), a nucleotide or protein length, an amount of binding, etc. is meant to encompass variations of ±20% or ±10%, more preferably ±5%, even more preferably ±1, and still more preferably ±0.1% from the specified amount, as such variations are appropriate to perform a disclosed method or otherwise carry out the present invention.

The term “substantially identical”, is used herein to describe a degree of similarity between nucleotide sequences, and refers to two or more sequences that have at least about least 60%, preferably at least about 70%, more preferably at least about 80%, more preferably about 90% to 99%, still more preferably about 95% to about 99%, and most preferably about 99% nucleotide identify, when compared and aligned for maximum correspondence, as measured using one of the following sequence comparison algorithms or by visual inspection. Preferably, the substantial identity exists in nucleotide sequences of at least about 100 residues, more preferably in nucleotide sequences of at least about 150 residues, and most preferably in nucleotide sequences comprising a full length coding sequence. The term “full length” is used herein to refer to a complete open reading frame encoding a functional T1R or T2R polypeptide, as described further herein below. Methods for determining percent identity between two polypeptides are defined herein below under the heading “Nucleotide and Amino Acid Sequence Comparisons”.

In one aspect, substantially identical sequences can be polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise one or more residue changes, a deletion of residues, or an insertion of additional residues.

Another indication that two nucleotide sequences are substantially identical is that the two molecules hybridize specifically to or hybridize substantially to each other under stringent conditions. In the context of nucleic acid hybridization, two nucleic acid sequences being compared can be designated a “probe” and a “target.” A “probe” is a reference nucleic acid molecule, and a “target” is a test nucleic acid molecule, often found within a heterogeneous population of nucleic acid molecules. A “target sequence” is synonymous with a “test sequence.”

A preferred nucleotide sequence employed for hybridization studies or assays includes probe sequences that are -complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of the particular T1R or T2R. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” and “stringent hybridization wash conditions” refer to conditions under which a probe will hybridize to its target subsequence, typically in a complex mixture of nucleic acids but to no other sequences. Stringent conditions are sequence-dependent and will be different in different circumstances. Longer sequences hybridize specifically at higher temperatures. An extensive guide to the hybridization of nucleic acids is that in Tigssen, Techniques in Biochemistry and Molecular Biology—Hybridization With Nucleic Probes, “overview of principles of hybridization and the strategy of nucleic acid assays.” (1973) Generally, highly stringent hybridization and wash conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium).

Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the additional of destabilizing agents such as formamide. For selective or specific hybridization, a positive signal is at least two times background, optionally 10 times background hybridization. Exemplary stringent hybridization conditions are:

50% formamide, 5×SSC, and 1% SDS, incubating at 42° C. or 5×SSC, 1% SDS, incubating at 65° C. The hybridization and wash steps effected in said exemplary stringent hybridization conditions are each effected for at least 1, 2, 5, 10, 15, 30, 60, or more minutes. Preferably, the wash and hybridization steps are each effected for at least 5 minutes, and more preferably, 10 minutes, 15 minutes, or more than 15 minutes.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y. for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are additional examples of hybridization and wash conditions that can be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 MM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1 SDS at 50° C.; more preferably, a probe and target sequence hybridize in 7% sodium dodecyl sulphate (SDS), 0.5M NaPO4, 1 EDTA at 50° C. followed by washing in 0.1 ×SSC, 0.1% SDS at 65° C.

A further indication that two nucleic acid sequences are substantially identical is that proteins encoded by the nucleic acids are substantially identical, share an overall three-dimensional structure, or are biologically functional equivalents. Nucleic acid molecules that do not hybridize to each other under stringent conditions are still substantially identical if the corresponding proteins are substantially identical. This can occur, for example, when two nucleotide sequences comprise conservatively substituted variants as permitted by the genetic code.

Nucleic acids that do not hybridize to each other under stringent conditions are still substantially related if the polypeptides that they encode are substantially related. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. Preferably, the wash and hybridization steps are each effected for at least 5 minutes. A positive hybridization is at least twice background. Those of ordinary skill will readily recognize that alternative hybridization and wash conditions can be utilized to provide conditions of similar stringency.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; and Rossolini et al. (1994) Mol Cell Probes 8:91-98.

The term T1R or T2R also encompasses nucleic acids comprising subsequences and elongated sequences of a T1R or T2R nucleic acid, including nucleic acids complementary to a T1R or T2R nucleic acid, T1R or T2R RNA molecules, and nucleic acids complementary to T1R or T2R RNAs (cRNAs).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences,” as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof A gene can be obtained by a variety of methods, including cloning from a biological sample, synthesis based on known or predicted sequence information, and recombinant derivation of an existing sequence.

The term “chimeric gene,” as used herein, refers to a promoter region operatively linked to a T1R or T2R sequence, including a T1R or T2R cDNA, a T1R or T2R nucleic acid encoding an antisense RNA molecule, a T1R or T2R nucleic acid encoding an RNA molecule having tertiary structure (e.g., a hairpin structure) or a T1R or T2R nucleic acid encoding a double-stranded RNA molecule. The term “chimeric gene” also refers to a T1R or T2R promoter region operatively linked to a heterologous sequence.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a T1R or T2R polypeptide, as described further herein below.

The term “construct”, as used herein to describe a type of construct comprising an expression construct, refers to a vector further comprising a nucleotide sequence operatively inserted with the vector, such that the nucleotide sequence is recombinantly expressed.

The terms “recombinantly expressed” or “recombinantly produced” are used interchangeably to refer generally to the process by which a polypeptide encoded by a recombinant nucleic acid is produced.

The term “heterologous nucleic acids” refers to a sequence that originates from a source foreign to an intended host cell or, if from the same source, is modified from its original form. Thus, preferably recombinant T1R or T2R nucleic acids comprise heterologous nucleic acids. A heterologous nucleic acid in a host cell can comprise a nucleic acid that is endogenous to the particular host cell but has been modified, for example by mutagenesis or by isolation from native cis-regulatory sequences. A heterologous nucleic acid also includes non-naturally occurring multiple copies of a native nucleotide sequence. A heterologous nucleic acid can also comprise a nucleic acid that is incorporated into a host cell's nucleic acids at a position wherein such nucleic acids are not ordinarily found.

Nucleic acids used in the cell-based assays of the present invention can be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) Molecular Cloning: A Laboratory Manual. Cold Spring Harbor Laboratory Press, Cold Spring Harbor, N.Y. (1989); Silhavy et al. Experiments with Gene Fusions. Cold Spring Harbor Laboratory Press, Cold Spring Harbor, N.Y. (1984); Glover & Hames DNA Cloning: A Practical Approach, 2nd ed. IRL Press and Oxford University Press, Oxford/New York (1995); Ausubel (ed.) Short Protocols in Molecular Biology, 3rd ed. Wiley, N.Y. (1995).

The term “substantially identical”, as used herein to describe a level of similarity between a particular T1R or T2R protein or oCNGC subunit and a protein substantially identical to the T1R or T2R or protein or oCNGC subunit, refers to a sequence that is at least about 35% identical to the particular T1R or T2R or oCNGC protein, when compared over the full length of the T1R or T2R or oCNGC protein. Preferably, a protein substantially identical to the T1R or T2R or oCNGC protein used in the present invention comprises an amino acid sequence that is at least about 35% to about 45% identical to a particular T1R or T2R or oCNGC subunit, more preferably at least about 45% to about 55% identical thereto, even more preferably at least about 55% to about 65% identical thereto, still more preferably at least about 65% to about 75% identical thereto, still more preferably at least about 75% to about 85% identical thereto, still more preferably at least about 85% to about 95% identical thereto, and still more preferably at least about 95% to about 99% identical thereto when compared over the full length of the particular T1R or T2R or oCNGC subunit. The term “full length” refers to a functional T1R or T2R or oCNGC polypeptide. Methods for determining percent identity between two polypeptides are also defined herein below under the heading “Nucleotide and Amino Acid Sequence Comparisons”.

A preferred modified oCNGC used in assays according to the invention will comprise an oCNGC comprising one or more mutations that render the oCNGC more sensitive to cAMP. For example, this application exemplifies a modified oCNGC wherein the OCNC1 subunit is mutated at two amino acids—Cys458Trp, Glu581Met. This mutated form is more sensitive to cAMP and enhances the sensitivity of oCNGC-based assays. (These mutations were previously reported in Rich et al., J. Gen. Physiol. 118: 63-77 (2001)).

The term “substantially identical,” when used to describe polypeptides, also encompasses two or more polypeptides sharing a conserved three-dimensional structure. Computational methods can be used to compare structural representations, and structural models can be generated and easily tuned to identify similarities around important active sites or ligand binding sites. See Saqi et al. Bioinformatics 15:521-522 (1999); Barton Acta Crystallogr D Biol Crystallogr 54:1139-1146 (1998); Henikoff et al. Electrophoresis 21:1700-1706 (2000); and Huang et al. Pac Symp Biocomput:230-241 (2000).

Substantially identical proteins also include proteins comprising amino acids that are functionally equivalent to a T1R or T2R according to the invention. The term “functionally equivalent” in the context of amino acids is known in the art and is based on the relative similarity of the amino acid side-chain substituents. See Henikoff & Henikoff Adv Protein Chem 54:73-97 (2000). Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., J. Mol. Biol. 157(1):105-32 (1982)). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of line amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The term “substantially identical” also encompasses polypeptides that are biologically functional equivalents of a particular T1R or T2R or oCNGC polypeptide. The term “functional” includes an activity of an T1R or T2R polypeptide, for example activating intracellular signaling pathways (e.g., coupling with gustducin) and mediating taste perception. Preferably, such activation shows a magnitude and kinetics that are substantially similar to that of a cognate T1R or T2R or oCNGC polypeptide in vivo. Representative methods for assessing T1R or T2R and oCNGC activity are described in the patent applications incorporated by reference and herein.

The assays of the present invention also can use functional fragments of a particular T1R or T2R polypeptide. Such functional portion need not comprise all or substantially all of the amino acid sequence of a native T1R or T2R gene product. The assays of the present invention also can use functional polypeptide sequences that are longer sequences than that of a native T1R or T2R polypeptide. For example, one or more amino acids can be added to the N-terminus or C-terminus of a T1R or T2R polypeptide. Such additional amino acids can be employed in a variety of applications, including but not limited to purification applications. Methods of preparing elongated proteins are known in the art.

“MAPK” or “SAP Kinase” refers to a mitogen activated protein kinase, the expression of which is activated by some functional GPCRs, i.e., T2Rs and T1Rs.

“MAPK” or “MAP Kinase” activation specific ligands” refers to a ligand, preferably a polyclonal or monoclonal antibody or fragment thereof that specifically binds an activated form of MAPK, e.g., p42/p44 MAPK or p38IMAPK Antibodies that specifically bind the activated (phosphorylated) form of MAPK are commercially available and include the phosph-p44tp42 MAP Kinase antibody #9106 available from Cell Signaling Technologies, the polyclonal anti-phospho-p44/42 MAPK and anti-phospho-p38 MAPK antibodies available from UBI, (Lake Placid, N.Y., USA) and New England Biolabs (Beverly, Mass., USA), the anti-phospho-p44142 MAPK antibodies reported by Discovery Research Laboratories III, Takeda Chemical Indust. Ltd., (Oskaka Japan) (Tan et al., J. Immunol. Meth. 232(1-2): 87-97 (1998)).

“PLC” refers to phospholipase C.

### oCNGC—Taste Receptor Cell-based Assays of the Present Invention

Thus, the present invention generally relates to cell-based assays for identifying compounds that modulate the activity of at least one T1R or T2R taste receptor, wherein the assays comprise contacting a eukaryotic cell that stably or transiently expresses at least one functional T1R or T2R, an olfactory CNG channel and a G protein that functionally couples therewith, e.g., a G protein such as Gα_{i/o} with a putative modulator of said functional T1R or T2R, and assaying the effect of said putative enhancer, agonist, antagonist or modulatory compound on the activity of said olfactory CNG channel, e.g., by monitoring changes in intracellular calcium.

The cells used in the subject assays, preferably eukaryotic cells, will stably or transiently express at least one functional T1R or T2R and a functional olfactory CNG channel preferably a human olfactory CNG channel. This eukaryotic cell will either stably or transiently express a functional T1R1/T1R3 umami taste receptor or a functional T1R2/T1R3 sweet taste receptor or will stably or transiently express at least one functional T2R. Also, preferably the functional T1R or T2R taste receptor will comprise a human T1R or T2R. Further such cells will also express a functional olfactory CNG channel, preferably human. Also, in order to produce a functional taste receptor, the eukaryotic cell will further be transfected to stably or transiently express or will endogenously express a G protein that couples with said T1R(s) or T2R thereby resulting in a functional taste receptor. Examples of suitable G proteins are known in the art and are referred in the patent applications incorporated by reference herein. In a preferred embodiment, the G protein will comprise a Gα_{i/o} protein selected from Gαᵢ, i.e. Gαᵢ₁₋₁, Gαᵢ₁₋₂, Gαᵢ₁₋₃, Gαᵢₒ₋₁, and Gαᵢₒ₋₂. Alternatively, such G proteins may include α-transducin, gustducin, G_{αz} or a functional chimera or variant thereof that couples with the T1R(s) or T2R expressed by the eukaryotic cell.

As noted, these cells will stably or transiently express at least one functional olfactory CNG channel, preferably a human olfactory CNG channel. Preferably, the cell line will be transfected or transformed with a nucleic acid sequence or sequence comprising several CNG subunits, e.g., OCNC1, OCNC2 and βb1. Sequences for human OCNC1, OCNC2 and OCNβb1 are contained in the Appendix of sequences that precedes the claims.

The present assays can be effected using any cell that functionally expresses the particular T1R(s) or T2R and an olfactory CNG channel and which cell, when contacted with a modulator of said T1R or T1R results in a detectable change in olfactory CNG channel activity, e.g., based on changes in intracellular calcium levels. Examples of suitable eukaryotic cells include amphibian, yeast, insect, amphibian, worm and mammalian cells. Specific examples of suitable cells for use in the subject cell-based assays include HEK, HEK-293T, HEK-293 cells, BHK cells, CHO cells, Hela cells, Cos cells, NIH3T3 cells, Swiss3T3 cells and Xenopus oocytes.

In a preferred embodiment the eukaryotic cells used in the subject cell-based assays, will comprise HEK-293 cells that stably or transiently express at least one or functional T1R or T2R taste receptor and a functional olfactory CNG channel by the transfection of such cells with a cDNA or cDNAs encoding oCNGC subunit sequences and a T1R or T2R and sequence which when expressed result in a functional olfactory CNG channel and T1R or T2R. For example, HEK-293 cells stably expressing the large T cell antigen and G_{αi} can be transiently transfected with a particular taste receptor plasmid and an oCNGC subunit plasmid by known transfection methods, e.g., by use of Ca²⁺ phosphate or lipid-based systems, or other transformation methods referenced supra. As noted previously, the T1R or T2R and olfactory CNG channel expressing cell will further express endogenously or be engineered to express a G protein that functionally couples with the T1R or T2R, e.g., a G protein selected from the G_{αi/o} proteins identified previously, such that activation of the taste receptor affects oCNGC activity.

Detailed Description of Olfactory CNG Channel Assays Used in the Invention to Indirectly Identify T1R or T2R Taste Modulators

The assays of the present invention measure changes in CNG channel activity or in the activity of proteins or second messengers associated with CNG channel activity, i.e., for the purpose of identifying ligands or screening small molecules to be used in blocking or enhancing taste modalities, i.e., sweet umami, sweet or bitter taste. For instance, the invention includes cation-based assays for monitoring changes in CNG channel activation comprising (1) introducing one or more nucleic acids encoding and expressing at least one human olfactory CNG channel subunit and at least one T1R or T2R sequence into host cells wherein said at least one CNG channel subunit forms a functional CNG channel; and (2) measuring changes in the amount of activation of said CNG channel in the presence and absence of different stimuli wherein said changes are measured via a change in the level of one or more intracellular cations. As described herein, functional CNG channels may be formed by the expression of all three subunits, or by expressing just OCNC1 and OCNC2, or by expressing OCNC1 alone. In this sense, “functional” means forming a channel through which extracellular cations may enter and changes in the level of intracellular cations due to CNG stimulation or inhibition may be measured and quantitated.

In such assays, at least one functional human olfactory CNG channel subunit is preferably encoded by a sequence selected from the group consisting of sequences that hybridize under high stringency conditions to the human OCNC sequence contained in the Appendix. This subunit can be expressed alone or in combination with other olfactory CNG channel subunits to form a functional CNG channel; i.e., a cation channel regulated by cyclic-nucleotides. For instance, the OCNC1 subunit may be expressed along with the OCNC2 and/or βb1 subunits, particularly those encoded by the nucleic acids of the invention that hybridize under high stringency conditions to the OCNC2 and βb1 sequences disclosed herein respectively. Orthologs of CNG channel subunits, i.e., OCNC2 and/or β1b subunits from other species, may also be expressed in the assays of the invention along with human CNG channel subunits, for instance, a human OCNC1 subunit, where the co-expression of such subunits forms a functional chimeric CNG channel. In a preferred embodiment these channel subunits may comprise one or more mutations that enhance the sensitivity of the oCNGC to CAMP.

In the subject assays, the host cells may be first stimulated with an agent that induces a basal level of CNG activation such as forskolin (or other activators of adenylyl cyclase), IBMX (or other inhibitors of cAMP phosphodiesterase). Olfactory CNG channel activation is then quantitated by monitoring ion flux into treated cells using fluorescent sodium chelators or fluorescent calcium chelators such as fura-2 (Abe et al., J. Biol Chem 267:13361-13368 (1992)); fluorescent sodium chelators such as sodium green tetraacetate (Molecular Probes) and Na.sup.+-Sensitive Dye Kit (Molecular Devices); or membrane potential dyes such as the Membrane Potential Dye Kit (Molecular Devices) and the Oxanol-Coumarin Kit (Aurora Biosciences). Olfactory CNG channel activators are identified by their ability to potentiate the fluorescent response to increased cAMP, and smell-blocking channel antagonists could be identified by their ability to attenuate the fluorescent response. The olfactory CNG channel could also be used as a surrogate for the identification of modulators of other CNG channels.

Assays for GPCRs and Other Proteins that Regulate cAMP Levels

The present invention includes assays for proteins that regulate cyclic-nucleotide levels. Such assays include those designed to measure changes in CNG channel activity resulting from changes in cyclic-nucleotide levels. Preferably, sensitized olfactory CNG channels that make use of subunit variants such as OCNC1[C458W/E581M] can be used to increase the sensitivity of these assays. Such assays can be used to quantitate the activity of GPCRs that couple to G proteins that regulate adenylyl cyclases or phosphodiesterases, and to identify GPCR modulators by high-throughput screening.

Particularly, a nucleic acid encoding a G-protein coupled taste receptor, i.e., T1R or T2R is introduced into the host cells used in the assays of the invention, in addition to nucleic acids encoding one or more CNG channel subunits and nucleic acids encoding G proteins if necessary. Sequences of potential T1Rs and T2Rs that my be used are provided in the Appendix of sequences that precedes the claims. In such an assay, stimuli could be screened for potential modulators of G-protein coupled receptor activity, via an affect of subsequent cyclic nucleotide levels on CNG channel activity.

In a preferred embodiment of the invention, such T1R or T2R taste receptor is first activated by exposure to a ligand, and said cells are screened for stimuli that further increase the activation of said receptor, thereby leading to a decrease in CNG activation (taste enhancer). Such enhancers could act at the level of the receptor to decrease CNG channel activation. Such enhancers could also act on adenylyl or guanylyl cyclase, phosphodiesterase, or any other protein that regulates cyclic nucleotide levels. Alternatively, such cells could be screened for stimuli that decrease the activation of the T1R or T2R receptor, thereby leading to an increase in CNG activation (i.e. taste blockers).

The invention also encompasses variations of any assay described herein further comprising the use of control cells. For instance, assays including expression of a desired G protein-coupled receptor could further comprise the steps of. (a) providing a second host cell that expresses said at least one CNG channel subunit so as to form a functional CNG channel but that does not express said G-protein coupled receptor; (b) measuring changes in the amount of activation of the CNG channel in said second host cell in the presence and absence of different stimuli; and (c) comparing said changes in the amount of activation of said CNG channel in said second host with the amount of activation of said CNG channel in said cell expressing said G-protein coupled receptor, i.e., a T1R or T2R.

The assays of the present invention particularly include high-throughput screening assays. Apparatuses for quantitating simultaneously measurements from a multitude of samples are known in the art. For example, a Fluorometric Imaging Plate Reader (FLIPR) is available from Molecular Devices, and may be used for single wavelength detection of changes in intracellular calcium or sodium, membrane potential and pH. The apparatus and reader can be programmed to simultaneously deliver compounds to and image all 96 or 384 wells of a microplate within one second, and is therefore amendable to high throughput formats. An argon-ion laser excites a fluorescent indicator dye suitable for the specific change being measured, and the emitted light is detected using the associated optical system. A camera system then images the entire plate and integrates data over a time interval specified by the user.

Alternatively, apparatuses such as the Voltage Ion Probe Reader (VIPR) of Aurora Biosciences may be used for dual wavelength detection of fluorescence resonance energy transfer (FREI) between two fluorescent molecules. FRET is a distance-dependent interaction between the electronic excited states of two dye molecules, and may be used to investigate a variety of biological events that produce changes in molecular proximity, including the activity of Na+, K+, Cl−, Ca2+, and Ligand-gated Ion Channels. Aurora Biosciences Corporation's voltage sensor probe technology uses FRET between a membrane-bound donor molecule and a mobile, voltage-sensitive, acceptor molecule to detect membrane potential. The VIPR reader is amenable to both 96- and 384-well formats.

The high-throughput assays of the invention include a variety of formats. For instance, one embodied high throughput assay for detecting or measuring the activity of a CNG channel in response to at least two or more potential test compounds in at least two or more individual compartments simultaneously, comprises (1) introducing one or more nucleic acids encoding and expressing at least one olfactory CNG channel subunit into suitable host cells wherein said at least one CNG channel subunit forms a functional CNG channel such that, when activated either directly or indirectly, said channel causes a change in the intracellular concentration of a predetermined ion; (2) transferring said host cells to a divided culture vessel having an array of individual compartments (either before or after transfection); (3) loading said host cells with an ion-sensitive fluorescent indicator sufficient to detect a change in the concentration of a predetermined ion, e.g., calcium or sodium; or changes in membrane potential (4) delivering to said at least two or more individual compartments one or more different test compounds or combination of test compounds wherein said test compounds have the potential either directly or indirectly to activate said CNG channel; and (5) detecting or measuring in at least two of said compartments the fluorescence emitted by the ion-sensitive indicator in order to detect a change in the concentration of the predetermined ion in response to potential activation of said CNG channel. While the assay may be used to simultaneously measure at least two samples as mentioned above, preferred high throughput formats preferably involve the screening of at least 5, more preferably 10, more preferably 50, more preferably 100, and possibly even hundreds or thousands of samples simultaneously. The number of samples screened in a single throughput may be based on the number of individual compartments in the particular plate to be used, i.e., 24, 96, 384, etc.

In a variation of the high throughput assays of the invention, at least one of the individual compartments in the array may be exposed to a known activator of CNG channels at the same time that said at least two compartments are exposed to said test compounds. Such a further compartment would serve as a positive control for the detection of CNG channel activity. Compartments containing suitable negative controls could also be included. Known activators of CNG channels that may be used for positive controls include forskolin, IBMX, permeant analogs of cAMP and cGMP, and nitric oxide (NO)-generating compounds (such as S-nitrosocysteine—SNC).

Any cell amenable to a high throughput format may used in the assays of the invention. Particularly preferred are cells that grow in a monolayer, as such cells may give more consistent results when used in a fluorescence plate reader. Suitable cells have been identified upon and include e.g., human embryonic kidney cells (HEK-293 cells), COS cells, mouse L cells, Swiss 3T3 cells, Chinese hamster ovary cells, African green monkey kidney cells, Ltk-cells, and BHK cells.

Isolation and Expression of T1R, T2R and CNG Channel Subunits

The subject cell-based assays require T1R and/or T2R and olfactory CNG channel subunit nucleic acid sequences.

Isolation and expression of the olfactory CNG channel subunits or fragments or variants thereof, used in the assays of the present invention can be performed as described below. PCR primers can be used for the amplification of nucleic acids encoding olfactory CNG channel subunits based on the sequence contained in FIG. 1 and libraries of these nucleic acids can thereby be generated. Libraries of expression vectors can then be used to infect or transfect host cells for the functional expression of these libraries. These genes and vectors can be made and expressed in vitro or in vivo. One of skill will recognize that desired phenotypes for altering and controlling nucleic acid expression can be obtained by modulating the expression or activity of the genes and nucleic acids (e.g., promoters, enhancers and the like) within the vectors of the invention. Any of the known methods described for increasing or decreasing expression or activity can be used. The invention can be practiced in conjunction with any method or protocol known in the art, which are well described in the scientific and patent literature.

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to mammalian cells, e.g., bacterial, yeast, insect or plant systems.

Alternatively, these nucleic acids can be synthesized in vitro by well-known chemical synthesis techniques, as described in, e.g., Carruthers, Cold Spring Harbor Symp. Quant. Biol. 47:411-418 (1982); Adams, Am. Chem. Soc. 105:661 (1983); Belousov, Nucleic Acids Res. 25:3440-3444 (1997); Frenkel, Free Radic. Biol. Med. 19:373-380 (1995); Blommers, Biochemistry 33:7886-7896 (1994); Narang, Meth. Enzymol. 68:90 (1979); Brown, Meth. Enzymol. 68:109 (1979); Beaucage, Tetra. Lett. 22:1859 (1981); U.S. Pat. No. 4,458,066. Double-stranded DNA fragments may then be obtained either by synthesizing the complementary strand and annealing the strands together under appropriate conditions, or by adding the complementary strand using DNA polymerase with an appropriate primer sequence.

Techniques for the manipulation of nucleic acids, such as, for example, for generating mutations in sequences, subcloning, labeling probes, sequencing, hybridization and the like are well described in the scientific and patent literature. See, e.g., Sambrook, ed., Molecular Cloning: a Laboratory manual (2nd ed.), Vols. 1-3, Cold Spring Harbor Laboratory (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I, Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immunoelectrophoresis, radioimmunoassay (RIA), enzyme-linked immunosorbent assay (ELISA), immunofluorescent assay, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Oligonucleotide primers are used to amplify nucleic acid encoding an olfactory CNG channel subunit. The nucleic acids described herein can also be cloned or measured quantitatively using amplification techniques. Also using exemplary degenerate primer pair sequences, the skilled artisan can select and design suitable oligonucleotide amplification primers. Amplification methods are also well known in the art, and include, e.g., polymerase chain reaction, PCR (PCR Protocols, a Guide to Methods and Applications, ed. Innis. Academic Press, N.Y., 1990 and PCR Strategies, ed. Innis, Academic Press, NY, 1995), ligase chain reaction (LCR) (see, e.g., Wu, Genomics 4:560, 1989; Landegren, Science 241:1077, 1988; Barringer, Gene 89:117, 1990); transcription amplification (see, e.g., Kwoh, Proc. Natl. Acad. Sci. USA 86:1173, 1989); and, self-sustained sequence replication (see, e.g., Guatelli, Proc. Natl. Acad. Sci. USA 87:1874, 1990); Q Beta replicase amplification (see, e.g., Smith, J. Clin. Microbiol. 35:1477, 1997); automated Q-beta replicase amplification assay (see, e.g., Burg, Mol. Cell. Probes 10:257, 1996) and other RNA polymerase mediated techniques (e.g., NASBA, Cangene, Mississauga, Ontario); see also Berger, Methods Enzymol. 152:307,1987; Sambrook; Ausubel; U.S. Pat. Nos. 4,683,195 and 4,683,202; Sooknanan, Biotechnology 13:563, 1995.

Once amplified, the nucleic acids, either individually or as libraries, may be cloned according to methods known in the art, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” the PCR primer pair.

Paradigms to design degenerate primer pairs are well known in the art. For example, a COnsensus-DEgenerate Hybrid Oligonucleotide Primer (CODEHOP) strategy computer program is known in the art and uses the BlockMaker multiple sequence alignment site for hybrid primer prediction beginning with a set of related protein sequences (see, e.g., Rose, Nucl. Acids Res. 26:1628, 1998; Singh, Biotechniques 24:318,1998).

Means to synthesize oligonucleotide primer pairs are well known in the art. “Natural” base pairs or synthetic base pairs can be used. For example, use of artificial nucleobases offers a versatile approach to manipulate primer sequence and generate a more complex mixture of amplification products. Various families of artificial nucleobases are capable of assuming multiple hydrogen bonding orientations through internal bond rotations to provide a means for degenerate molecular recognition. Incorporation of these analogs into a single position of a PCR primer allows for generation of a complex library of amplification products. See, e.g., Hoops, Nucleic Acids Res. 25:4866, 1997. Nonpolar molecules can also be used to mimic the shape of natural DNA bases. A non-hydrogen-bonding shape mimic for adenine can replicate efficiently and selectively against a nonpolar shape mimic for thymine (see, e.g., Morales, Nat. Struct. Biol. 5:950, 1998). For example, two degenerate bases can be the pyrimidine base 6H, 8H-3,4-dihydropyrimido[4,5c][1,2]oxazin-7-one or the purine base N6-methoxy-2,6-diaminopurine (see, e.g., Hill, Proc. Natl. Acad. Sci. USA 95:4258, 1998). Exemplary degenerate primers of the invention incorporate the nucleotide analog 5′-Dimethoxytrityl-N-benzoyl-2′-deoxy-Cytidine, 3′-[(2-cyanoethyl)-(N,N-d-iisopropyl)]-phosphoramidite (the term “P” in the sequences, This pyrimidine analog hydrogen bonds with purines, including A and G residues.

Nucleic acids that encode olfactory CNG channel subunits or are generated by amplification (e.g., PCR) of appropriate nucleic acid sequences using degenerate primer pairs. In the case of CNG channel subunits the amplified nucleic acid can be genomic DNA from any cell or tissue or mRNA or cDNA derived from olfactory receptor-expressing cells, e.g., olfactory neurons or olfactory epithelium. T1R or T2R sequences can be similarly isolated from taste cells or synthesized.

Isolation of DNAs from olfactory cells and taste cells is well known in the art, as discussed above. For example, cells can be identified by olfactory marker protein (OMP), an abundant cytoplasmic protein expressed almost exclusively in mature olfactory sensory neurons (see, e.g., Buiakova, Proc. Natl. Acad. Sci. USA 93:9858, 1996). Shirley, Eur. J. Biochem. 32:485,1983), describes a rat olfactory preparation suitable for biochemical studies in vitro on olfactory mechanisms. Cultures of adult rat olfactory receptor neurons are described by Vargas, Chem. Senses 24:211, 1999). Also, U.S. Pat. No. 5,869,266 describes culturing human olfactory neurons for neurotoxicity tests and screening. Murrell, J. Neurosci. 19:8260, 1999), describes differentiated olfactory receptor-expressing cells in culture that respond to odorants, as measured by an influx of calcium.

Hybrid protein-coding sequences comprising the subject human CNG channel subunits or T1R or T2R sequences can also be fused to the translocation sequences. Also, these nucleic acid sequences can be operably linked to transcriptional or translational control elements, e.g., transcription and translation initiation sequences, promoters and enhancers, transcription and translation terminators, polyadenylation sequences, and other sequences useful for transcribing DNA into RNA. In construction of recombinant expression cassettes, vectors, transgenics, and a promoter fragment can be employed to direct expression of the desired nucleic acid in all tissues. Olfactory cell-specific transcriptional elements can also be used to express the fusion polypeptide receptor, including, e.g., a 6.7 kb region upstream of the M4 olfactory receptor coding region. This region was sufficient to direct expression in olfactory epithelium with wild type zonal restriction and distributed neuronal expression for endogenous olfactory receptors (Qasba, J. Neurosci. 18:227, 1998). Receptor genes are normally expressed in a small subset of neurons throughout a zonally restricted region of the sensory epithelium. The transcriptional or translational control elements can be isolated from natural sources, obtained from such sources as ATCC or GenBank libraries, or prepared by synthetic or recombinant methods.

Fusion proteins, either having C-terminal or, more preferably, N-terminal translocation sequences, may also comprise the translocation motif described herein. However, these fusion proteins can also comprise additional elements for, e.g., protein detection, purification, or other applications. Detection and purification facilitating domains include, e.g., metal chelating peptides such as polyhistidine tracts or histidine-tryptophan modules or other domains that allow purification on immobilized metals; maltose binding protein; protein A domains that allow purification on immobilized immunoglobulin; or the domain utilized in the FLAGS extension/affinity purification system (Immunex Corp, Seattle Wash.).

The inclusion of a cleavable linker sequences such as Factor Xa (see, e.g., Ottavi, Biochimie 80:289, 1998), subtilisin protease recognition motif (see, e.g., Polyak, Protein Eng. 10:615, 1997); enterokinase ([nvitrogen, San Diego, Calif.), and the like, between the translocation domain (for efficient plasma membrane expression) and the rest of the newly translated polypeptide may be useful to facilitate purification. For example, one construct can include a nucleic acid sequence encoding a polypeptide linked to six histidine residues followed by a thioredoxin, an enterokinase cleavage site (see, e.g., Williams, Biochemistry 34:1787, 1995), and an amino terminal translocation domain. The histidine residues facilitate detection and purification while the enterokinase cleavage site provides a means for purifying the desired protein(s) from the remainder of the fusion protein. Technology pertaining to vectors encoding fusion proteins and application of fusion proteins are well described in the scientific and patent literature, see, e.g., Kroll, DNA Cell. Biol. 12:441, 1993).

Expression vectors, either as individual expression vectors or as libraries of expression vectors, comprising the olfactory binding domain-encoding sequences may be introduced into a genome or into the cytoplasm or a nucleus of a cell and expressed by a variety of conventional techniques, well described in the scientific and patent literature. See, e.g., Roberts, Nature 328:731, 1987; Berger supra; Schneider, Protein Expr. Purif. 6435:10, 1995; Sambrook; Tijssen; Ausubel. Product information from manufacturers of biological reagents and experimental equipment also provide information regarding known biological methods. The vectors can be isolated from natural sources, obtained from such sources as ATCC or GenBank libraries, or prepared by synthetic or recombinant methods.

The nucleic acids can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required. For example, the marker may encode antibiotic resistance (e.g., chloramphenicol, kanamycin, G418, bleomycin, hygromycin) or herbicide resistance (e.g., chlorosulfuron or Basta) to permit selection of those cells transformed with the desired DNA sequences (see, e.g., Blondelet-Rouault, Gene 190:315, 1997; Aubrecht, J. Pharmacol. Exp. Ther. 281:992, 1997). Because selectable marker genes conferring resistance to substrates like neomycin or hygromycin can only be utilized in tissue culture, chemoresistance genes are also used as selectable markers in vitro and in vivo.

The present invention also includes not only the DNA and proteins having the specified amino acid sequences, but also DNA fragments, particularly fragments of, for example, 40, 60, 80, 100, 150, 200, or 250 nucleotides, or more, as well as protein fragments of, for example, 10, 20, 30, 50, 70, 100, or 150 amino acids, or more.

Also contemplated are chimeric proteins, comprising at least 10, 20, 30, 50, 70, 100, or 150 amino acids, or more, of at least one of the sensory receptor human CNG channel subunits described herein, and optionally other peptides, e.g., another receptor subunit or a reporter polypeptide. Chimeric receptors are well known in the art, and the techniques for creating them and the selection and boundaries of domains or fragments of different receptors are also well known. Thus, this knowledge of those skilled in the art can readily be used to create such chimeric receptors. The use of such chimeric receptors can provide, for example, an olfactory selectivity characteristic of one of the receptors specifically disclosed herein, coupled with the signal transduction characteristics of another receptor, such as a well known receptor used in prior art assay systems.

Polymorphic variants, alleles, and interspecies homologs that are substantially identical to a human olfactory CNG subunit disclosed herein can be isolated using nucleic acid probes constructed based on the sequences contained in FIG. 1. Alternatively, expression libraries can be used to isolate sensory receptors and polymorphic variants, alleles, and interspecies homologs thereof, by detecting expressed homologs immunologically with antisera or purified antibodies made against a sensory receptor-derived polypeptide, which also recognize and selectively bind to the sensory receptor homolog.

Also within the scope of-the invention are cells for use in the assays of the present invention which express human CNG channel subunit fragments, or variants, T1R or T2R sequences or functional fragments or variants and a suitable G protein. To obtain high levels of expression of a cloned gene or nucleic acid, such as cDNAs encoding the CNG subunit fragments, or variants thereof, the nucleic acid sequence of interest is subcloned into an expression vector that contains a strong promoter to direct transcription, a transcription/translation terminator, and if for a nucleic acid encoding a protein, a ribosome binding site for translational initiation. Suitable prokaryotic and eukaryotic expression systems are well known in the art and described, e.g., in Sambrook et al.

Any of the well-known procedures for introducing foreign nucleotide sequences into host cells may be used. These include the use of calcium phosphate transfection, polybrene, protoplast fusion, electroporation, liposomes, microinjection, plasmid vectors, viral vectors and any of the other well known methods for introducing cloned genomic DNA, cDNA, synthetic DNA or other foreign genetic material into a host cell (see, e.g., Sambrook et al.). It is only necessary that the particular genetic engineering procedure used be capable of successfully introducing at lest one gene into the host cell capable of expressing the olfactory receptor, fragment, or variant of interest.

After the expression vector is introduced into the cells, the transfected cells are cultured under conditions favoring expression of the receptor, fragment, or variant of interest, which is then recovered from the culture using standard techniques. Examples of such techniques are well known in the art. See, e.g., WO 00/06593, which is incorporated by reference in a manner consistent with this disclosure.

Other Cell-Based Functional Assays

In the preferred embodiment, at least one CNG channel subunit polypeptide and a functional T1R or T2R is expressed in a eukaryotic cell. Preferably, HEK-293 cells, and activation of such channels in such cells can be detected, e.g., based on changes in intracellular Ca⁺⁺ or Na⁺.

Samples or assays that are treated with a potential inhibitor or activator are compared to control samples without the test compound, to examine the extent of modulation. Such assays may be carried out in the presence of a compound that is known to activate the T1R or T2R. Control samples (untreated with activators or inhibitors) are assigned a relative sensory receptor activity value of 100. Receptor activation is achieved when the channel activity value relative to the control is about 90%, optionally 50%, optionally 25-0%. Receptor inhibition is achieved when the channel activity value relative to the control is 110%, optionally 150%, 200-500%, or 1000-2000%.

Changes in ion flux may also be assessed by determining changes in polarization (i.e., electrical potential) of the cell or membrane expressing a T1R or T2R and oCNGC. One means to determine changes in cellular polarization is by measuring changes in current, and thereby measuring changes in polarization, with voltage-clamp and patch-clamp techniques, e.g., the “cell-attached” mode, the “inside-out” mode, and the “whole cell” mode (see, e.g., Ackerman et al., New Engl. J Med., 336:1575, 1997). Whole cell currents are conveniently determined using the standard. Other known assays include: assays to measure ion flux using radiolabeled or fluorescent probes such as voltage-sensitive dyes (see, e.g., Vestergarrd-Bogind et al., J. Membrane Biol., 88:67, 1988; Gonzales & Tsien, Chem. Biol., 4:269, 1997; Daniel et al., J. Pharmacol. Meth., 25:185, 1991; Holevinsky et al., J. Membrane Biology, 137:59, 1994) or ion sensitive dyes, e.g., calcium or sodium sensitive dyes such as fluo-3, fluo-4, or fura-2. Generally, the compounds to be tested are present in the range from 1 pM to 100 mM.

In another embodiment, transcription levels can be measured to assess the effects of a test compound on signal transduction. A host cell containing a CNG channel subunit protein of interest , which optionally is mutated to enhance cAMP sensitivity is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the channel subunit protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, luciferase, 3′-galactosidase and alkaline phosphatase. Furthermore, the channel subunit protein of interest can be used as an indirect reporter via attachment to a second reporter such as green fluorescent protein (see, e.g., Mistili & Spector, Nature Biotech. 15:961, 1997).

The amount of transcription is then compared to the amount of transcription in either the same cell in the absence of the test compound, or it may be compared with the amount of transcription in a substantially identical cell that lacks the channel subunit protein of interest. A substantially identical cell may be derived from the same cells from which the recombinant cell was prepared but which had not been modified by introduction of heterologous DNA. Any difference in the amount of transcription indicates that the test compound has in some manner altered the activity of the CNG channel.

Modulators

The compounds tested as modulators of a T1R or T2R based on their effect on the activity of olfactory CNG channel can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing. of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487, 1991; and Houghton et al., Nature 354:84, 1991). Other chemistries for generating chemical diversity libraries can also be used. Such chemistries include, but are not limited to: peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiaze-pines and dipeptides (Hobbs et al., Proc. Nat. Acad. Sci. 90:6909, 1993), vinylogous polypeptides (Hagihara et al., J. Amer. Chem. Soc. 114:6568, 1992), nonpeptidal peptidomimetics with glucose scaffolding (Hirschmann et al., J. Amer. Chem. Soc. 114:9217, 1992), analogous organic syntheses of small compound libraries (Chen et al., J. Amer. Chem. Soc. 116:2661, 1994), oligocarbamates (Cho et al., Science 261:1303, 1993), peptidyl phosphonates (CAMPbell et al., J. Org. Chem. 59:658, 1994), nucleic acid libraries (Ausubel, Berger and Sambrook, all supra), peptide nucleic acid libraries (U.S. Pat. No. 5,539,083), antibody libraries (Vaughn et al., Nature Biotechnology 14:309, 1996 and WO 97/00271), carbohydrate libraries (Liang et al., Science 274:1520, 1996) and U.S. Pat. No. 5,593,853), small organic molecule libraries (benzodiaze-pines, Baum, C&EN, page 33, Jan. 18, 1993); thiazolidinones and metathiazanones, U.S. Pat. No. 5,549,974; pynrolidines, U.S. Pat. Nos. 5,525,735 and 5,519,134; morpholino compounds, U.S. Pat. No. 5,506,337; benzodiazepines, U.S. Pat. No. 5,288,514, and the like.

Devices for the preparation of combinatorial libraries are commercially available (see, e.g., 357 MPS, 390 MPS (Advanced Chem Tech, Louisville, Ky.), Symphony (Rainin, Woburn, Mass.), 433A (Applied Biosystems, Foster City, Calif.), 9050 Plus (Millipore, Bedford, Mass.)). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences; Columbia, Md.; etc.).

The present invention also provides for kits for screening for novel taste modulators based on their effect on the function of olfactory CNG channels. Such kits can be prepared from readily available materials and reagents, as well as any of the aforementioned products. For example, such kits can comprise any one or more of the following materials: CNG channel encoding nucleic acids or proteins, T1R and/or T2R encoding nucleic acids or proteins, or test cells that co-express such sequences, reaction tubes, and instructions for testing CNG channel activity. A wide variety of kits and components can be prepared according to the present invention, depending upon the intended user of the kit and the particular needs of the user.

Cells that stably or transiently express the particular taste receptor and oCNGC can also be used in assays that measure the effect of at least one putative T1R or T2R modulatory compound on other G_{αi/o}-mediated signaling pathways, e.g., by measuring its effect on MAPK activation, cAMP accumulation or adenylyl cyclase activity. The MAPK or cAMP assays of the present invention can use immobilized cells or cells in suspension. In a preferred embodiment taste cells will be seeded into multi-well culture plates, e.g., 6-well culture plates. However, other in vitro cell culture devices can be substituted therefore, and is not critical to the invention.

In a typical MAPK or cAMP assay, functional expression of the T1R or T2R expressing eukaryotic cell is allowed to proceed for a certain time, e.g., on the order of about 48 hours, and then taste receptor expressing cells are stimulated with a putative modulatory compound for a fixed time, e.g., about 5 minutes, and then the reaction is then stopped, e.g., by the addition of ice-cold buffer, and the cells are then assayed for changes in activated MAPK, cAMP or adenylyl cyclase activity. However, these reaction times may be shortened or lengthened within wide limits.

The level of activated MAPK, cAMP or adenylyl cyclase produced by such cells is detected in whole cells or cell lysates. In a preferred embodiment, cell lysates are prepared by known methods, and detected by activated cAMP, MAPK or adenylyl cyclase activity is detected by known methods. For example, activated MAPK can be detected by use of a polyclonal or monoclonal antibody or fragment thereof that specifically recognizes an activated (phosphorylated) form of MAPK.

### Additional Exemplification of Other Cell-Based Assays

The following are exemplary of other types of cell-based assays that may additionally be used according to the invention for detecting the effect of a putative modulator on T1R or T2R activity.

### 1. GTP Assay

For GPCRs T1R OR T2R, a measure of receptor activity is the binding of GTP by cell membranes containing receptors. In the method described by Traynor and Nahorski, 1995, Mol. Pharmacol. 47: 848-854, (1995) one essentially measures G-protein coupling to membranes by detecting the binding of labelled GTP. For GTP binding assays, membranes isolated from cells expressing the receptor are incubated in a buffer containing 20 mM HEPES, pH 7.4, 100 mM NaCl, and 10 mM MgCl₂, 80 pM .³⁵S-GTPγS and 3 μM GDP.

The assay mixture is incubated for 60 minutes at 30° C., after which unbound labelled GTP is removed by filtration onto GF/B filters. Bound, labelled GTP is measured by liquid scintillation counting. The presence and absence of a candidate modulator of T1R or T2R activity. A decrease of 10% or more in labelled GTP binding as measured by scintillation counting in an assay of this kind containing a candidate modulator, relative to an assay without the modulator, indicates that the candidate modulator inhibits T1R or T2R activity. A compound is considered an agonist if it induces at least 50% of the level of GTP binding when the compound is present at 1 μM or less.

GTPase activity is measured by incubating the membranes containing a T1R or T2R polypeptide with γ³²P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not expressing T1R or T2R (mock-transfected), in order to exclude possible non-specific effects of the candidate compound.

In order to assay for the effect of a candidate modulator on T1R or T2R-regulated GTPase activity, membrane samples are incubated with and without the modulator, followed by the GTPase assay. A change (increase or decrease) of 10% or more in the level of GTP binding or GTPase activity relative to samples without modulator is indicative of T1R or T2R modulation by a candidate modulator.

### 2. Downstream Pathway Activation Assays

i) Calcium Flux—The Aequorin-Based Assay:

The aequorin assay takes advantage of the responsiveness of mitochondrial apoaequorin to intracellular calcium release induced by the activation of GPCRs (Stables et al., Anal. Biochem. 252:115-126 (1997); Detheux et al., 2000, J. Exp. Med., 192 1501-1508 (2000); both of which are incorporated herein by reference). Briefly, T1R or T2R-expressing clones are transfected to coexpress mitochondrial apoaequorin and G_{α16}. Cells are incubated with 5 μM Coelenterazine H (Molecular Probes) for 4 hours at room temperature, washed in DMEM-F12 culture medium and resuspended at a concentration of 0.5.times.10.sup.6 cells/ml. Cells are then mixed with test agonist molecules and light emission by the aequorin is recorded with a luminometer for 30 seconds. Results are expressed as Relative Light Units (RLU). Controls include assays using membranes isolated from cells not expressing T1R or T2R (mock transfected), in order to exclude possible non-specific effects of the candidate compound.

Aequorin activity or intracellular calcium levels are “changed” if light intensity increases or decreases by 10% or more in a sample of cells, expressing a T1R or T2R polypeptide and treated with a candidate modulator, relative to a sample of cells expressing the T1R or T2R polypeptide but not treated with the candidate modulator or relative to a sample of cells not expressing the T1R or T2R polypeptide (mock-transfected cells) but treated with the candidate modulator.

(ii) Adenylate Cyclase Assay:

Assays for adenylate cyclase activity are described by Kenimer & Nirenberg, Mol. Pharmacol. 20: 585-591 (1981). That assay is a modification of the assay taught by Solomon et al., 1974, Anal. Biochem. 58: 541-548 (1974), also incorporated herein by reference. Briefly, 100l1 reactions contain 50 mM Tris-Hcl (pH 7.5), 5 mM MgCl₂, 20 mM creatine phosphate (disodium salt), 10 units (71 μg of protein) of creatine phosphokinase, 1 mM α-_{32P} (tetrasodium salt, 2 μCᵢ), 0.5 mM cyclic AMP, G-^{3H}-labeled cyclic AMP (approximately 10,000 cpm), 0.5 mM Ro2O-1724, 0.25% ethanol, and 50-200 μg of protein homogenate to be tested (i.e., homogenate from cells expressing or not expressing a T1R or T2R polypeptide, treated or not treated with a candidate modulator). Reaction mixtures are generally incubated at 37° C. for 6 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a T1R or T2R polypeptide.

Adenylate cyclase activity is “changed” if it increases or decreases by 10% or more in a sample taken from cells treated with a candidate modulator of T1R or T2R activity, relative to a similar sample of cells not treated with the candidate modulator or relative to a sample of cells not expressing the T1R or T2R polypeptide (mock-transfected cells) but treated with the candidate modulator.

(iii) cAMP Assay:

Intracellular or extracellular cAMP is measured using a CAMP radioimmunoassay (RIA) or CAMP binding protein according to methods widely known in the art. For example, Horton & Baxendale, Methods Mol. Biol. 41: 91-105 (1995), which is incorporated herein by reference, describes an RIA for CAMP.

A number of kits for the measurement of CAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of CAMP is “changed” if the level of CAMP detected in cells, expressing a T1R or T2R polypeptide and treated with a candidate modulator of T1R or T2R activity (or in extracts of such cells), using the RIA-based assay of Horton & Baxendale, 1995, increases or decreases by at least 10% relative to the CAMP level in similar cells not treated with the candidate modulator.

(iv) Phospholipid Breakdown, DAG Production and Inositol Triphosphate Levels:

Receptors that activate the breakdown of phospholipids can be monitored for changes due to the activity of known or suspected modulators of T1R or T2R by monitoring phospholipid breakdown, and the resulting production of second messengers DAG and/or inositol triphosphate (IP₃). Methods of detecting each of these are described in Phospholipid Signaling Protocols, edited by Ian M. Bird. Totowa, N.J., Humana Press, (1998), which is incorporated herein by reference. See also Rudolph et al., J. Biol. Chem. 274: 11824-11831 (1999) (137), which also describes an assay for phosphatidylinositol breakdown. Assays should be performed using cells or extracts of cells expressing T1R or T2R, treated or not treated or without a candidate modulator. Control reactions should be performed using mock-transfected cells, or extracts from them in order to exclude possible non-specific effects of some candidate modulators.

According to the invention, phosphatidylinositol breakdown, and diacylglycerol and/or inositol triphosphate levels are “changed” if they increase or decrease by at least 10% in a sample from cells expressing a T1R or T2R polypeptide and treated with a candidate modulator, relative to the level observed in a sample from cells expressing a T1R or T2R polypeptide that is not treated with the candidate modulator.

(v) PKC Activation Assays:

Growth factor receptor tyrosine kinases can signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341 (1982), can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/2 mM DTT immediately prior to assay.

The substrate for the assay is the peptide Ac-FKKSFKL-NH₂, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ. Cofactors required for the assay include calcium, magnesium, ATP, phosphatidylserine and diacylglycerol. Depending upon the intent of the user, the assay can be performed to determine the amount of PKC present (activating conditions) or the amount of active PKC present (non-activating conditions). For most purposes according to the invention, non-activating conditions will be used, such that the PKC, that is active in the sample when it is isolated, is measured, rather than measuring the PKC that can be activated. For non-activating conditions, calcium is omitted from the assay in favor of EGTA.

The assay is performed in a mixture containing 20 mM HEPES, pH 7.4, 1-2 mM DIT, 5 mM MgCl₂, 100 μM ATP, .about. 1 μCᵢ .γ³²P-ATP, 100 μg/ml peptide substrate (^{˜}100 μM), 140 μM/3.8 μM phosphatidylserine/diacylglycerol membranes, and 100 μM calcium (or 500 μM EGTA). 48 μL of sample, diluted in 20 mM HEPES, pH 7.4, 2 mM DTT is used in a final reaction volume of 80 μl. Reactions are performed at 30° C. for 5-10 minutes, followed by addition of 25 μl of 100 mM ATP, 100 mM EDTA, pH 8.0, which stops the reactions.

After the reaction is stopped, a portion (85 μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 min. Bound radioactivity is measured by scintillation counting. Specific activity (cpm/nmol) of the labelled ATP is determined by spotting a sample of the reaction onto PS1 paper and counting without washing. Units of PKC activity, defined as nmol phosphate transferred per min, are then calculated by known methods.

An alternative assay can be performed using a Protein Kinase C Assay Kit sold by PanVera (Cat. # P2747).

Assays are performed on extracts from cells expressing a T1R or T2R polypeptide, treated or not treated with a candidate modulator. Control reactions should be performed using mock-transfected cells, or extracts from them in order to exclude possible non-specific effects of some candidate modulators.

According to the invention, PKC activity is “changed” by a candidate modulator when the units of PKC measured by either assay described above increase or decrease by at least 10%, in extracts from cells expressing T1R or T2R and treated with a candidate modulator, relative to a reaction performed on a similar sample from cells not treated with a candidate modulator.

(iv) Kinase Assays:

MAP Kinase assays have already been described supra. MAP kinase activity can be assayed using any of several kits available commercially, for example, the p38 MAP Kinase assay kit sold by New England Biolabs (Cat # 9820) or the FlashPlate™ MAP Kinase assays sold by Perkin-Elmer Life Sciences.

MAP Kinase activity is “changed” if the level of activity is increased or decreased by 10% or more in a sample from cells, expressing a T1R or T2R polypeptide, treated with a candidate modulator relative to MAP kinase activity in a sample from similar cells not treated with the candidate modulator.

Direct assays for tyrosine kinase activity using known synthetic or natural tyrosine kinase substrates and labelled phosphate are well known, as are similar assays for other types of linases (e.g., Ser/Thr kinases). Kinase assays can be performed with both purified kinases and crude extracts prepared from cells expressing a T1R or T2R polypeptide, treated with or without a candidate modulator. Control reactions should be performed using mock-transfected cells, or extracts from them in order to exclude possible non-specific effects of some candidate modulators. Substrates can be either full-length protein or synthetic peptides representing the substrate. Pinna & Ruzzene (Biochem. Biophys. Acta 1314: 191-225 (1996)) list a number of phosphorylation substrate sites useful for detecting kinase activities. A number of kinase substrate peptides are commercially available. One that is particularly useful is the “Src-related peptide,” RRLIEDAEYAARG (available from Sigma # A7433), which is a substrate for many receptor and nonreceptor tyrosine kinases. Because the assay described below requires binding of peptide substrates to filters, the peptide substrates should have a net positive charge to facilitate binding. Generally, peptide substrates should have at least 2 basic residues and a free amino terminus. Reactions generally use a peptide concentration of 0.7-1.5 mM.

Assays are generally carried out in a 25 μl volume comprising 5 .mu.l of 5× kinase buffer (5 mg/mL BSA, 150 mM Tris-Cl (pH 7.5), 100 mM MgCl₂; depending upon the exact kinase assayed for, MnCl₂ can be used in place of or in addition to the MgCl₂), 5 .mu.l of 1.0 mM ATP (0.2 mM final concentration), γ³²P-ATP (100-500 cpm/pmol), 3 μl of 10 mM peptide substrate (1.2 mM final concentration), cell extract containing kinase to be tested (cell extracts used for kinase assays should contain a phosphatase inhibitor (e.g., 0.1-1 mM sodium orthovanadate)), and H₂0 to 25pl. Reactions are performed at 30° C., and are initiated by the addition of the cell extract.

Kinase reactions are performed for 30 seconds to about 30 minutes, followed by the addition of 45 μl of ice-cold 10% trichloroacetic acid (TCA). Samples are spun for 2 minutes in a microcentrifuge, and 35 μl of the supernatant is spotted onto Whatman P81 cellulose phosphate filter circles. The filters are washed three times with 500 ml cold 0.5% phosphoric acid, followed by one wash with 200 ml of acetone at room temperature for 5 minutes. Filters are dried and incorporated ³²P is measured by scintillation counting. The specific activity of ATP in the kinase reaction (e.g., in cpm/pmol) is determined by spotting a small sample (2-5 μl) of the reaction onto a P81 filter circle and counting directly, without washing. Counts per minute obtained in the kinase reaction (minus blank) are then divided by the specific activity to determine the moles of phosphate transferred in the reaction.

Tyrosine kinase activity is “changed” if the level of kinase activity is increased or decreased by 10% or more in a sample from cells, expressing a T1R or T2R polypeptide, treated with a candidate modulator relative to kinase activity in a sample from similar cells not treated with the candidate modulator.

(vii) Transcriptional Reporters for Downstream Pathway Activation:

The intracellular signal initiated by binding of an agonist to a receptor, e.g., T1R or T2R, sets in motion a cascade of intracellular events, the ultimate consequence of which is a rapid and detectable change in the transcription or translation of one or more genes. The activity of the receptor can therefore be monitored by detecting the expression of a reporter gene driven by control sequences responsive to T1R or T2R activation.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription-based reporter assay provides a rapid indication of whether a given receptor is activated.

Reporter genes such as luciferase, CAT, GFP, β-lactamase or β-galactosidase are well known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful for making reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other. known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., Cell 51: 513-514) (1987) : a TATA box that is required for transcription initiation; two upstream elements for basal transcription, and an enhancer, which includes an element with dyad symmetry and which is required for induction by TPA, serum, EGF, and PMA.

The 20 bp c-fos transcriptional enhancer element located between −317 and −298 bp upstream from the c-fos MRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by detecting either the binding of the transcription factor, or the expression of a reporter gene linked to a CREB-binding element (termed the CRE, or cAMP response element). The DNA sequence of the CRE is TGACGTCA. (Reporter constructs responsive to CREB binding activity are described in U.S. Pat. No. 5,919,649).

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666) (1988); the somatostatin gene promoter (cAMP responsive; Montminy et al., Proc. Natl. Acad. Sci. 83:6682-6686 (1986)); the proenkephalin promoter (responsive to cAMP, nicotinic agonists, and phorbol esters; Comb et al., Nature 323:353-356 (1986)); the phosphoenolpyruvate carboxy-kinase (PEPCK) gene promoter (cAMP responsive; Short et al., J. Biol. Chem. 261:9721-9726 (1986)).

Additional examples of transcriptional control elements that are responsive to changes in GPCR activity include, but arc not limited to those responsive to the AP-1 transcription factor and those responsive to NF-KB activity. The consensus AP-1 binding site is the palindrome TGA(C/G)TCA (Lee et al., Nature 325: 368-372 (1987); Lee et al., Cell 49: 741-752 (1987)). The AP-1 site is also responsible for mediating induction by tumor promoters such as the phorbol ester 12-O-tetradecanoylphorbol-.beta.-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP-1 activity), Fos-related antigens (Fra) 1 and 2, I κβα, ornithine decarboxylase, and annexins I and II.

The NF-KB binding element has the consensus sequence GGGGACTTTCC. A large number of genes have been identified as NF-KB responsive, and their control elements can be linked to a reporter gene to monitor GPCR activity. A small sample of the genes responsive to NF-KB includes those encoding IL-1β. (Hiscott et al., Mol. Cell. Biol. 13:6231-6240 (1993) (148)), TNF-α (Shakhov et al., J. Exp. Med. 171: 35-47 (1990)), CCR5 (Liu et al., AIDS Res. Hum. Retroviruses 14: 1509-1519 (1998)), P-selectin (Pan & McEver, J. Biol. Chem. 270: 23077-23083 (1995)), Fas ligand (Matsui et al., J. Immunol. 161: 3469-3473 (1998)), GM-CSF (Schreck & Baeuerle, Mol. Cell. Biol. 10: 1281-1286 (1990)) and κβα (Haskill et al., Cell 65: 1281-1289 (1991)). Vectors encoding NF-KB-responsive reporters are also known in the art or can be readily made by one of skill in the art using, for example, synthetic NF-KB elements and 20 a minimal promoter, or using the NF-KB-responsive sequences of a gene known to be subject to NF-KB regulation. Further, NF-KB responsive reporter constructs are commercially available e.g., from CLONTECH.

To screen for agonists, the cells are left untreated, exposed to candidate modulators, and expression of the reporter is measured. An increase of at least 50% in reporter expression in the presence of a candidate modulator indicates that the candidate is a modulator of T1R or T2R activity. An agonist will induce at least as many, and preferably the same amount or more of reporter expression than buffer alone. This approach can also be used to screen for inverse agonists where cells express a T1R or T2R polypeptide at levels such that there is an elevated basal activity of the reporter. A decrease in reporter activity of 10% or more in the presence of a candidate modulator, relative to its absence, indicates that the compound is an inverse agonist.

To screen for antagonists, the cells expressing T1R or T2R and carrying the reporter construct are contacted in the presence and absence of a candidate modulator. A decrease of 10% or more in reporter expression in the presence of candidate modulator, relative to the absence of the candidate modulator, indicates that the candidate is a modulator of T1R or T2R activity.

Controls for transcription assays include cells not expressing T1R or T2R but carrying the reporter construct, as well as cells with a promoterless reporter construct. Compounds that are identified as modulators of T1R or T2R-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate T1R or T2R activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the MRNA content of one cell or tissue, in the different libraries used for screening of T1R or T2R.

(viii) Inositol Phosphate (IP) Measurement:

Cells of the invention are labelled for 24 hours with 10 μCi/ml³H] inositol in inositol free DMEM containing 5% FCS, antibiotics, amphotericin, sodium pyruvate and 400 μg/ml G418. Cells are incubated for 2 h in Krebs-Ringer Hepes (KRH) buffer of the following composition (124 mM NaCl, 5 mM KCl, 1.25 mM MgSO₄, 1.45 mM CaCl₂, 1.25 mM KH₂PO₄, 25 mM Hepes (pH:7.4) and 8 mM glucose). The cells are then challenged with various nucleotides for 30 s. The incubation is stopped by the addition of an ice cold 3% perchloric acid solution. IP are extracted and separated on Dowex columns as previously described. 2MeSATP and ATP solutions (1 mM) are treated at room temperature with 20 units/ml CPK and 10 Mm cp for 90 min to circumvent problems arising from the contamination and degradation of triphosphate nucleotide solutions.

T1R or T2R Assay

The invention may further include an assay for detecting the activity of a receptor of the invention in a sample. For example, T1R or T2R activity can be measured in a sample comprising a cell or a cell membrane that expresses T1R or T2R. The assay is performed by incubating the sample in the presence or absence of a modulator and carrying out a second messenger assay, as described above. The results of the second messenger assay performed in the presence or absence of the activator are compared to determine if the T1R or T2R receptor is active.

Any of the assays of receptor activity, including but not limited to the GTP-binding, GTPase, adenylate cyclase, cAMP, phospholipid-breakdown, diacylglycerol, inositol triphosphate, arachidonic acid release (see below), PKC, kinase and transcriptional reporter assays, can be used to determine the presence of an agent in a sample, e.g., a tissue sample, that affects the activity of the T1R or T2R receptor molecule. To do so, T1R or T2R polypeptide is assayed for activity in the presence and absence of the sample or an extract of the sample. An increase in T1R or T2R activity in the presence of the sample or extract relative to the absence of the sample indicates that the sample contains an agonist of the receptor activity. A decrease in receptor activity in the presence of an agonist and the sample, relative to receptor activity in the absence thereof, indicates that the sample contains an antagonist of T1R or T2R activity.

The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation.

Other functional assays include, for example, microphysiometer or biosensor assays (see Hafner, 2000, Biosens. Bioelectron. 15: 149-158) (2000)).

### Functional Coupling of Gα_{i/o} Proteins to T1Rs and T2Rs

As earlier mentioned, the present invention relates to Applicants' previous discovery that T1Rs and T2Rs functionally couple to G proteins other than promiscuous G proteins such as Gα₁₅ or gustducin. Particularly, this invention relates to Applicant's previous discovery that T1Rs and T2Rs functionally couple to Gα_{i/o} proteins and use Gα_{i/o} to transmit signals to downstream effectors, e.g., cAMP, adenylyl cyclase and MAP Kinase.

Gₛ stimulates the enzyme adenylyl cyclase . By contrast, Gᵢ (and G_{z} and Gₒ) inhibit this enzyme. Adenylyl cyclase catalyzes the conversion of ATP to cAMP. Thus, constitutively activated GPCRs that couple Gᵢ (or G_{z} and Gₒ) protein associated with a decrease in cellular levels of cAMP. See, generally, “Indirect Mechanisms of Synoptic Transmission,” Chapter 8, From Neuron to Brain (3rd Edition), Nichols, J. G. et al eds., Sinaver Associates, Inc. (1992). Thus, assays that detect cAMP can be utilized to determine if a compound is e.g., an inverse agonist to the receptor (i.e., such a compound would increase the levels of cAMP): As earlier explained, a variety of approaches can be used to measure cAMP, e.g., anti-cAMP antibodies in an ELISA method, or the second messenger reporter system assays described supra.

A Gᵢ protein coupled receptor is known to inhibit adenylyl cyclase, resulting in a decrease in cAMP production. Another effective technique for measuring the decrease in production of cAMP as an indication of constitutive activation of a receptor that predominantly couples Gᵢ upon activation can be accomplished by co-transfecting a signal enhancer, e.g., a non-endogenous, constitutively activated receptor that predominantly couples with Gₛ upon activation with the Gᵢ linked GPCR, i.e., a T1R or T2R. In contrast to Gᵢ coupled GPCRs, constitutive activation of a Gₛ coupled receptor can be determined based upon an increase in production of cAMP. Thus, this construction approach is intended to advantageously exploit these “opposite” effects. For example, co-transfection of a non-endogenous, constitutively activated Gₛ coupled receptor (“signal enhancer”) with the Gᵢ coupled receptor (T1R or T2R) provides a baseline cAMP signal (i.e., although the Gᵢ coupled receptor will decrease cAMP levels, this “decrease” will be relative to the substantial increase in cAMP levels established by constitutively activated Gₛ coupled signal enhancer). By then co-transfecting the signal enhancer with a constitutively activated version of the target receptor, cAMP will decrease further (relative to the baseline) due to the increased functional activity of the Gᵢ target, i.e., T1R or T2R, which decreases CAMP.

Screening for potential T1R or T2R modulators using such a CAMP assay can then be accomplished with two provisos: first, relative to the Gi coupled target receptor (T1R or T2R), “opposite” effects will result, i.e., an inverse agonist of the Gi coupled target receptor will decrease this signal; second candidate modulators that are identified using this approach should be assessed independently to ensure that these compounds do not target the signal enhancing receptor (this can be accomplished prior to or after screening against co-transfected receptor).

Additionally, as described above, other assays can be designed which assess the effects of CAMP on other cellular events. Alteration of the intracellular concentration of CAMP is known to affect many cellular reactions. For example, an increase in CAMP intracellular concentrations stimulates the activity of protein Kinases. For a general review of CAMP and secondary messenger systems associated therewith, reference is made to “Molecular Cell Biology”, Darnell et al, Chapter 16 (1986).

Particular signal substances that use CAMP as a second messenger include by way of example calcitonin, chorionic gonadotropin, corticotrophin, epinephrine, follicle-stimulating homone, glucagon, leutenizing hormone, lipotropin, melanocyte-stimulating hormone, norepinephrine, parathyroid hormone (PTH), thyroid-stimulating hormone and vasopressin.

The subject assays which measure the effect of a putative modulator or TR/Gᵢ associated signaling pathways were not suggested prior to Applicant's prior discovery that T1Rs and T2Rs used Gᵢ signaling pathways. In vivo, receptors for bitter and sweet taste functionally couple to the taste-specific G-protein α-gustducin to initiate the transduction cascade leading to taste perception. In heterologous cells, however, previously there was no direct evidence of functional coupling to G-proteins other than Gα₁₅, a promiscuous G-protein widely used for receptor deorphaning. Unexpectedly, the present inventors have earlier demonstrated that receptors for bitter, sweet and also umami taste couple effectively to Gᵢ-signaling pathways when expressed in human embryonic kidney cells. For example, as shown in Applicants' earlier application, cycloheximide, a bitter compound, specifically activates ERK1/2 mitogen-activated kinases in cells expressing the mouse bitter receptor mT2R05 and :the rat bitter receptors rT2R9, and that activation of ERK1/2 is totally abolished upon treatment with pertussis toxin indicating that these receptors couple to ERK1/2 activation through Gαᵢ. Also in agreement with these observations, cycloheximide inhibits the forskolin-induced cAMP accumulation in mT2R05-expressing cells by 70%. It was also shown in Applicants' earlier application that natural and artificial sweeteners such as sucrose, D-tryptophan, saccharin and cyclamate (known activators of T1R2/T1R3 sweet receptors) activate ERK1/2 in cells expressing the human sweet receptor hT1R2/hT1R3, that monosodium glutamate exclusively activates ERK1/2 in cells expressing the human umami receptor hT1R1/hT1R3 and that the effect thereof is greatly enhanced by the presence of inosine monophosphate, and consistent with Gi coupling, that these responses are prevented by treatment with pertussis toxin.

Still further, Applicant's showed previously that sweeteners including cyclamate, aspartame, saccharin, and monellin significantly inhibit the forskolin-induced CAMP accumulation in hT1R2/hT1R3-expressing cells (by 50-70%), and that monosodium glutamate similarly decreases basal levels of CAMP in hT1R1/hT1R3-expressing cells (by 50%).

### Applications of the Subject Assays

The present invention provides cell-based assay methods that rely on the discovery that T1Rs and T2Rs functionally couple to Gᵢ proteins e.g., Gαᵢ and transmit signals to downstream effectors, e.g., CAMP, MAP Kinase, and adenylyl cyclase that enable the identification of modulators, e.g., agonists, antagonists, inverse agonists enhancers of a T1R or T2R polypeptide. The T2R modulators of the invention are useful for altering taste perception, for example to induce, suppress or enhance bitter taste perception in a subject. The T1R2/T1R3 modulators are useful for modulating sweet taste, e.g., by enhancing the taste of another sweet tasting compound such as saccharin. The T1R1/T1R3 modulators identified according to the invention are useful for modulating umami taste, e.g., by enhancing the taste of a umami compound such as monosodium glutamate.

### Compositions

In accordance with the methods of the present invention, a composition that is administered to alter taste perception in a subject will comprise an effective amount of a T1R or T2R modulator (agonist, antagonist, or enhancer). A T1R or T2R activator or modulator can comprise any substance e.g., small molecule, peptide, protein, carbohydrate, oligosaccharide, glycoprotein, amino acid derivative, and the like. In general, compounds will be identified by screening libraries of potential taste modulatory compounds, which may be comprised of synthetic or naturally occurring compounds. The library may be random or may comprise compounds having related structures or are structures or substitutions. After lead candidates are identified, compound libraries having similar structure will be produced and screened for T1R or T2R modulatory activity according to the invention. T1R or T2R modulators identified as disclosed herein can be used to prepare compositions suitable for oral use, including but not limited to food, beverages, oral washes, dentifrices, cosmetics, and pharmaceuticals. T1R or T2R modulators can also be used as additives to alter the sweet, umami or bitter taste of a compound that is of palatable but undesirable for oral use, for example compounds comprised in household cleansers, poisons, etc. Such modulators will alter bitter, sweet or umami tasting compounds contained therein.

For example, representative foods having an undesirable or bitter taste include, but are not limited to, citrus fruits such as grapefruit, orange, and lemon; vegetables such as tomato, pimento, celery, melon, carrot, potato, and asparagus; seasoning or flavoring materials such as flavor, sauces, soy sauce, and red pepper; foods originating from soybean; emulsion foods such as cream, dressing, mayonnaise, and margarine; processed marine products such as fish meat, ground fish meat, and fish eggs; nuts such as peanuts; fermented foods such as fermented soybean; meats and processed meats; pickles; noodles; soups including powdery soups; dairy products such as cheese; breads and cakes; confectioneries such as candies, chewing gum, and chocolate; and specifically prepared foods for health.

Representative. cosmetics eliciting bitter taste (e.g., skin lotions, creams, face packs, lip sticks, foundations, shaving preparations, after-shave lotions, cleansing foams, and cleansing gels) include but are not limited to those compositions that include surfactants such as sodium alkyl sulfate and sodium monoalkyl phosphate; fragrances such as menthol, linalool, phenylethyl alcohol, ethyl propionate, geraniol, linalyl acetate and benzyl acetate; antimicrobials such as methyl paraben, propyl paraben and butyl paraben; humectants such as lactic acid and sodium lactate; alcohol-denaturating agents such as sucrose octaacetate and brucine; and astringents such as aluminum lactate.

Representative pharmaceuticals having a bitter taste include acetaminophen, terfenadine, guaifenesin, trimethoprim, prednisolone, ibuprofen, prednisolone sodium phosphate, methacholine, pseudoephedrine hydrochloride, phenothiazine, chlorpromazine, diphenylhydantoin, caffeine, morphine, demerol, codeine, lomotil, lidocaine, salicylic acid, sulfonamides, chloroquine, a vitamin preparation, minerals and penicillins. neostigmine, epinephrine, albuterol, diphenhydramine, chlorpheniramine maleate, chlordiazepoxide, amitriptyline, barbiturates, diphenylhydantoin, caffeine, morphine, demerol. codeine, lomotil, lidocaine, salicylic acid, sulfonamides, chloroquine, a vitamin preparation, minerals and penicillins.

Representative sweeteners which may be modulated by compounds according to the invention include xylitol, sorbitol, saccharin, sucrose, glucose, fructose, cyclamate, aspartame, monellin, and the like, and derivatives thereof.

Representative umami compounds, the taste which may be modulated according to the invention include L-glutamate, L-asparate, monosodium glutamate, derivatives thereof, compounds containing and the like.

These taste modulators can also be administered as part of prepared food, beverage, oral wash, dentifrice, cosmetic, or drug. To prepare a composition suitable for administration to a subject, a T1R or T2R modulator can be admixed with a compound, the taste of which is to be modulated in amount comprising about 0.001% to about 10% by weight, preferably from about 0.01% to about 8% by weight, more preferably from about 0.1% to about 5% by weight, and most preferably from about 0.5% to about 2% by weight.

Suitable formulations include solutions, extracts, elixirs, spirits, syrups, suspensions, powders, granules, capsules, pellets, tablets, and aerosols. Optionally, a formulation can include a pharmaceutically acceptable carrier, a suspending agent, a solubilizer, a thickening agent, a stabilizer, a preservative, a flavor, a colorant, a sweetener, a perfume, or a combination thereof. T1R or T2R modulators and compositions can be presented in unit-dose or multi-dose sealed containers, such as ampules and vials.

### Administration

T1R or T2R modulators can be administered directly to a subject for modulation of taste perception. Preferably, a modulator of the invention is administered orally or nasally.

In accordance with the methods of the present invention, an effective amount of a T1R or T2R modulator is administered to a subject. The term “effective amount” refers to an amount of a composition sufficient to modulate T1R or T2R activation and/or to modulate taste perception, e.g., bitter, sweet or umami taste perception.

An effective amount can be varied so as to administer an amount of an T1R or T2R modulator that is effective to achieve the desired taste perception. The selected dosage level will depend upon a variety of factors including the activity of the T1R or T2R modulator, formulation, combination with other compositions (e.g., food, drugs, etc.), the intended use (e.g., as a food additive, dentifrice, etc.), and the physical condition and prior medical history of the subject being treated.

An effective amount or dose can be readily determined using in vivo assays of taste perception as are known in the art. Representative methods for assaying taste perception are described infra.

The following examples are illustrative of the present invention and should not be interpreted as limiting the applicable scope of the invention in any way.

### EXAMPLES

The invention is further illustrated by the following non-limiting examples wherein the following materials and methods are used.

### Materials and Methods

Sweeteners, agonists and toxins. Sucrose, aspartame, cyclamate, monellin, monosodium glutamate, inosine monophosphate, isoprdterenol, epidermal growth factor, denatonium benzoate, quinine sulfate, cycloheximide, rolipram and forskolin were from Sigma (St-Louis, Mo.). Pertussis toxin (PTX) was from List Biological Laboratories (CAMPbell, Calif.).

Establishment of stable cell lines. An inducible expression system can be to produce a umami taste receptor line (hT1R1/hT1R3). Vectors are prepared using the GeneSwitch inducible system ([nvitrogen, Carlsbad, Calif.). hT1R1 and hT1R3 vectors were prepared by cloning receptor cDNA into pGeneIV5-His A at EcoRI/Not I sites. A modified pSwitch vector was also prepared by replacing the hygromycin β resistance gene with the puromycin resistance gene. The cDNAs for hT1R1, hT1R3, and puromycin resistance are co-transfected into HEK-293 cells stably expressing Gα₁₅ (Aurora Biosciences, San Diego, Chandraskekar et al, Cell 100(6): 703-11 (2000). hT1R1/hT1R3 stable cell lines are selected and maintained in high-glucose DMEM media containing 100 μg/mL zeocin, 0.5 μg/mL puromycin, 2 mM GIutaMAX 1, 10% dialyzed fetal bovine serum, 3 μg/mL blasticidin and penicillinistreptomyocin. To improve cell adhesion, cell flasks are pre-coated with Matrigel (Becton-Dickinson, Bedford, Mass.) at a dilution of 1:400. Expression of hT1R1 and hT1R3 is induced by treatment of cells with 6×10⁻¹¹ M mifepristone for 48 hours prior to experiments. Clones are tested and selected for mifepristone-induced responsiveness to MSG/IMP using calcium-imaging experiments.

Establishment of the sweet (hT1R2/R3) receptor line stable cell line is according to Li et al., Proc. Natl Acad. Sci, USA 99(7): 4692-6 (2002). Cells are maintained in low-glucose DMEM media containing 10% heat-inactivated dialyzed FIBS, penicillinlstreptomyocin, 3 μg/mL blasticidin, 100 ug/ml zeocin, and 0.5 ug/ml puromycin in Matrigel-coated flasks.

HEK-293 cells were transfected with 5 μg of linearized Rho-mT2R05 plasmid (Chandraskekar et al (2000)) in pEAK10 (Edge biosystems) using the Transit transfection reagent (Panvera). Cells were selected in the presence of 0.5 μg/ml puromycin, clones were isolated, expanded and analyzed by fluorescence-activated cell sorting for the presence of Rho tag immunoreactivity at the cell surface using a monoclonal antibody; raised against the first 40 amino acids of rhodopsin (Chandrashekar et al (2000); Adamus et al., Vision Res. 31(1): 17-31 (1991)).

Cloning Human Olfactory CNG Channel Subunits

Human orthologs of the three rat subunits disclosed by Thurauf et al. Eur. J. Neurosc. 8:2080-9 (1996) are contained in FIGS. 1-4. The presence of OCNC1 and OCNC2 in human olfactory epithelial mRNA by RT-PCR, and the full-length cDNAs for these two subunits were cloned by a combination of PCR from commercially available cDNA and PCR amplification of 5′ coding exons from genomic DNA. Terminal 5′ AscI and 3′ NotI sites were added flanking the human OCNC1 coding sequence, and the OCNC1 AscI-NotI fragment was cloned into the pEAK10 expression vector (Edge Biosystems) to produce plasmid SAV1931. The 5′ AscI site incorporated a 3 nucleotide linker sequence to introduce an optimized translation initiation site: GGCGCGCCgccATG, where the AscI site and start ATG are in UPPERCASE and the three-nucleotide linker is in lowercase.

The 3′ NotI site was added directly after the stop codon. Human OCNC2 was cloned analogously to produce plasmid SAV1976. Human β1b was cloned similarly to produce plasmid SAV2498; however, the 3 nucleotide linker separating the 5′ AscI site and the start ATG was omitted.

### Example 1

High-Throughput Assay for Monitoring Activity of the Human Olfactory CNG Channel

HEK-293T cells were transiently transfected with cloned human olfactory CNG channel subunits using lipid-based protocols. Transfection efficiencies, which were monitored by cotransfection of an RFP expression vector, were typically greater than 70%. After 24 hours, cells were harvested and transferred to 96 well plates. After an additional 24 hours, cells were loaded with either a fluorescent calcium or membrane potential dye for one hour, washed, and transferred to a FLIPR automated fluorometric plate reader with on-board fluidics.

In contrast to mock-transfected cells, cells transfected with olfactory CNG channel subunits displayed increased fluorescence following forskolin stimulation. Moreover, the magnitude of the forskolin response was subunit dependent: OCNC1 alone produced an active CNG channel, and OCNC2 (to a lesser extent) and Bulb (to a greater extent) potentiated OCNC1 activity (FIG. 5). The membrane potential dye provided a better signal to noise ratio than the calcium dye; however, assays using either dye are sufficiently robust for high-throughput screening (FIGS. 6 and 7).

### Example 2

CNG Channel-Based Fluorescence Assay for GPCRs.

Rich et al. (2001) constructed and characterized a mutant form of the rat OCNC1 CNG channel that has increased cAMP sensitivity. We generated the corresponding hOCNC1[C460W/E583M] mutant—carried in plasmid SAV2480—and found that in combination with β1b it has increased cAMP sensitivity and robust activity (FIG. 8). This increased sensitivity suggested that this human olfactory CNG channel variant might function as a CAMP biosensor and allow the development of whole cell-based fluorescence assays for GPCRs and other proteins that regulate CAMP levels.

HEK-293 cells were transfected with OCNC1[C458W/E581FM], OCNC2, and β1band a mouse olfactory receptor, mOREG, that is activated by eugenol (Kajiya et al., 2001). Transfected cells were seeded onto multi-well plates. After 48 hours, cells were loaded with a calcium dye for one hour, then washed, and their response to eugenol stimulation was monitored by fluorescence microscopy. Eugenol elicited fluorescence increases in transfected cells; this response likely reflects the eugenol-dependent activation of endogenous G.sub.s and adenylyl cyclase by mOREG because comparison to cells transfected with the CNG channel alone or mOREG alone established that these responses were CNG channel dependent and mOREG dependent (FIG. 9).

### Example 3

Development of a Cell Line that Stably Expresses the Human Olfactory CNG Channel Subunits OCNC1, OCNC2 and [symbol] 1b.

HEK-293 cells were transfected with the three human olfactory CNG channel subunits using lipid-based protocols. The appropriate selection antibiotics were added 72 hours after transfection and single colonies were recovered during a 3-5 week long period after the selection process. Colonies were screened for activity by transient transfection of the mOREG gene and the calcium influx was monitored after stimulation with the mOREG ligand eugenol.

The calcium image assay revealed several clones of cells transfected with the human CNG channel subunits that were responsive to eugenol. In contrast, mock transfected cells had no detectable eugenol response. Expression and activity remain stable after more than 20 passages. Moreover, cells stably expressing the human olfactory CNG subunits are more sensitive to stimulation to eugenol (FIG. 10). Therefore, cell lines which express these sequences are suitable for cell-based assays of CNG-mediated calcium transport.

### Example 4

Development of a Cell Line that Stably Expresses the Human Olfactory CNG Channel Subunits OCNC1 [C458W/E581M] and β1b.

HEK-293 cells were transfected with the two human olfactory CNG channel subunits OCNC and β1busing lipid-based protocols. The appropriate selection antibiotics were added 72 hours after transfection and single colonies were recovered during a 3-5 week long period after the selection process. Colonies were screened for activity by transientley transfecting the mOREG gene into the cells and monitoring the calcium influx when cells were stimulated whit the mOREG ligand eugenol.

The calcium image assay revealed several clones of cells transfected with the human CNG channel subunits that were responsive to eugenol. In contrast, mock transfected cells had no detectable eugenol response. Expression and activity remain stable after more than 20 passages. In contrast to cells transfected with wild type CNG subunits, the OCNC1 [C458W/E581M] transfected cells displayed significantly higher response to eugenol. Therefore, the cell lines developed are suitable for cell-based assays of CNG-mediated calcium transport. Moreover, the increased sensitivity makes the cell line amenable for screens for low affinity agonists or antagonists.

### Example 5

High-Throughput Assay Platform for Stably Expressed Human Olfactory CNG Channel Subunits.

The HEK-293 cells that stably express the human olfactory CNG channel were seeded into FLIPR imaging plates 24 hours prior to the experiment, on plates pre-coated with matrigel. The cells were loaded with a fluorescent calcium dye for one hour, washed and transferred to a FLIPR automated fluorometric plate reader with on-board fluidics. In contrast to the parent cells, cells stably expressing the human olfactory CNG channel subunits displayed increased fluorescence following stimulation of the .beta.2 receptor using isoproterenol (FIG. 11a). Moreover, the cells also showed increased fluorescence following stimulation with the adenylyl cyclase activator forskolin (FIG. 11b). Therefore, the cell-based assay is amenable to high throughput applications.

### Example 6

High-Throughput Assay Platform for an Activity-Enhanced, Stably Expressed Human Olfactory CNG Channel.

The HEK-293 cells that stably express the activity-enhanced human olfactory CNG channel were seeded into FLIPR imaging plates 24 hours prior to the experiment, on plates pre-coated with matrigel. The cells were loaded with a fluorescent calcium dye for one hour, washed and transferred to a FL]PR automated fluorometric plate reader with on-board fluidics. In contrast to the parent cells and cells transfected with wild type CNG subunits, cells stably expressing the activity-enhanced human olfactory CNG channel subunit displayed increased fluorescence following stimulation with the adenylyl cyclase activator forskolin (FIG. 11b). Moreover, the cells showed fluorescence similar to that observed with wild type CNG subunits following stimulation of the β2 receptor using isoproterenol (FIG. 11a). Therefore, the cell-based assay is amenable to high throughput applications, especially with respect to low affinity modulators.

### Example 7

### Effect of Isouroterenol Concentration on Calcium Influx in HEK-293—oCNGC Cells

Modulation of oCNG activity by taste receptors is measured in a cell-based assay that detects changes in intracellular calcium (as described in earlier examples). In brief, human embryonic kidney cells stably expressing the OCNC1 and are β1b subunits of oCNGC are transiently or stably transfected with T1R or T2R receptor plasmids. Transfected cells are seeded into 384-well culture plates, and functional expression is allowed to proceed for about 24 to 48 hours. The cells are then incubated with a calcium specific fluorescent dye (Fluo-4 or Fura-2, available from Molecular Probes) that provides for fast, simple and reliable fluorimetric detection of changes in calcium concentration within the cell.

As shown by the experimental results in FIG. 12, it was revealed that the addition of isoproterenol to the HEK-293—oCNGC taste receptor-expressing cells elicited a signaling cascade resulting in the activation of adenylyl cyclase and the accumulation of CAMP within the cells. This accumulation of CAMP induces oCNGC activation, which in turn causes extracellular calcium to flow inside the cells, resulting in a net increase of intracellular calcium concentration and a parallel increase of the fluorescence signal in the cells (FIG. 12).

More particularly, it was revealed that increasing concentrations of isoproterenol-induced calcium influx in HEK-293—oCNGC cells in a dose-dependent manner. (See FIG. 12).

### Example 8

### Effect of Increasing Concentrations of Sweeteners on Isoproterenol-Induced Calcium Influx in HEK 293—oCNGC Cells Expressing hT1R2/hT1R3

An experiment was conducted to assess the effect of various sweeteners (known to activate the human T1R2/T1R3 sweet receptor) on isoproterenol-induced calcium influx in HEK-293—oCNGC cells that express the human T1R2/T1R3 sweet receptor. As shown by the results in FIG. 13, increasing concentrations of sweetener inhibits isoproterenol-induced calcium influx in these cells. (In the figure, Delta F/F values were normalized to the fluorescence obtained after stimulation with 200 nM isoproterenol and each value corresponds to the means ± SD of a triplicate determination).

### Example 9

### Effect of Sweeteners on Isouroterenol-Induced Calcium Influx in Untransfected HEK 293—oCNGC Cells

An experiment was conducted to confirm that the effects of the sweeteners on calcium influx requires the expression of the sweet receptor. As shown in FIG. 14 isoproterenol-induced calcium influx is unaffected by the sweetener tested (aspartame, cyclamate, saccharin and neotame) in HEK-293—oCNG cells that do not express the hT1R2/hT1R3 sweet receptor.

In FIG. 14, it is shown that increasing concentrations of the tested sweeteners had no inhibitory effect on isoproterenol-induced calcium influx in untransfected HEK 293—oCNGC cells. Again, Delta F/F values were normalized to the fluorescence obtained after stimulation with 200 nM isoproternol and each value component to the mean ± SD of a triplicate determination).

### Example 10

### Effect of Pertusis Toxin on Sweetner-Induced Inhibition of Calcium Influx in HEK-293—oCNGC Cells Expressing hT1R2/hT1R3

An experiment was conducted to assess the effect of pertussis toxin (Plx) on the inhibition of isoproternol-ionduced calcium influx by a series of sweetener (aspartame, cyclamate, saccharin, neotame). As shown by the results in FIG. 15, pretreatment of these cells with PTx prevented the inhibition of isoproterenol-induced calcium influx by all sweeteners tested. These results confirm that the human T1R2/T1R3 sweet taste receptor couples to the inhibition of oCNGC through activation of G_{αi/o} proteins in HEK-293 cells. (This result is in accord with Applicants' earlier discovery disclosed in U.S. Ser. No. 10/770,127 that T1Rs and T2Rs functionally couple to Gα_{i/0} proteins).

### Example 11

### Effect of Cycloheximide on Isoproterenol-Induced Calcium Influx in HEK-293—oCNGC Cells Expressing a Mouse Bitter Taste Receptor

An experiment also was conducted to assess the effect of stimulation of the cycloheximide bitter taste receptor, mT2R05, on the isoproterenol-induced calcium influx in HEK-293—oCNGC cells expressing mT2R05. The results shown in FIG. 16 demonstrated that cycloheximide similar to examples using sweeteners described above inhibited isoproterenol-induced calcium influx.

Analogous to the previous examples, PTx prevented the inhibition of the isoproterenol-induced calcium influx by cycloheximide in HEK-293—oCNGC cells expressing mT2R05. More specifically, it was shown that PTx prevented inhibition of calcium inluux even with increasing concentrations of cycloheximide in HEK-293—oCNGC cells expressing mT2R05. As in figures above, Delta F/F values were normalized to the fluorescence obtained after stimulation with 200 nM isoproterenol. Each value corresponded to the mean +1-SD of a triplicate determination. Cells were also treated with PTx as in prior example. Under these conditions, cycloheximide failed to inhibit isoproterenol-induced calcium influx. (See FIG. 16). As anticipated, mT2R05 untransfected cells did not respond to cycloheximide. (FIG. 16).

Thus, the results in the foregoing examples provide compelling proof that both T1R and T2R taste receptors couple to the inhibition of oCNGC via activation of G_{αi/o} proteins in HEK-293 cells. This is in accord with Applicants' earlier discovery that T1R and T2R taste receptors functionally couple via G_{═i/o} proteins.

These experimental results establish that screening assays that select for T1R and T2R modulatory compounds can be designed that indirectly screen for T1R and T2R modulators based on the effect thereof on oCNGC activity in eukarytics cell lines that co-express a desired T1R or T2R, an oCNGC, and a G_{αi/o} protein. Particularly, assays may be designed that identify bitter, sweet and flavory (umami) receptor agonists, antagonists, enhancers and modulators, based on their effect on oCNGC activity, e.g., by flourimetrically monitoring changes in intracellular calcium concentration.

### Example 12

### Identification of GPCR Modulators Using CNG Channel Cell Line Containing the Human T1R2/T1R3 Sweet Taste G Protein-Coupled Receptor

Methods: A cell line, CNG136, co-expressing the human CNG channel and human T1R2/T1R3 sweet taste GPCR. This cell line was derived from HEK293 cells. CNG136 was tested with several sweet receptor agonists and modulators including, Compound 6542888, Compound 3069733, and Aspartame. Response to 10 uM of 6542888 and 3069733 and 5 mM Aspartame was measured using a FLIPR384 instrument (Molecular Devices) and is reported in Table I below. Data were normalized to the maximum activity obtained with a saturating concentration of Aspartame (5 mM). Compounds were also tested on control (“Parental CNG”) cells lacking the human sweet GPCR. Activity values correspond to an average of 2 independent determinations.

TABLE I
Results

| | % Max Activity | % Activity CNG |
|---|---|---|
| Compound | CNG136 | (Parental Cells) |
| 3069733 | 82 | 3 |
| 6542888 | 82 | −8 |
| Aspartame | 100 | 0 |

Summary: The results demonstrate that the two sweet taste receptor agonists and Aspartame, a known sweetener, each activated CNG136 but had no effect on the parental cell line expressing CNG alone in the absence of the sweet taste GPCR. The activity of the compounds for the sweet taste receptor using the CNG-based assay system was similar to that obtained using G15-based system, which couples via phospholipase Cbeta.

### CONCLUSIONS

In Applicant's earlier application, the present inventors investigated the functional coupling of taste receptors to ERK1/2 activation and to the modulation of intracellular CAMP levels, two classical signaling events activated by dozens of GPCRs (Morris et al., Physiol. Rev. 79(4): 1373-1430 (1999); Chin et al., Ann. NY Acad. Sci. 968: 49-64 (2002); Liebmann et al, J. Biol Chem. 271(49): 31098-31105 (1996)). cAMP is a universal second messenger used by a plethora of cell surface receptors to relay signals from the extracellular milieu to the intracellular signaling machinery such as protein kinases, transcription factors and ion channels (Morris and Malbon (1999); Chin et al (2002); Robinson-White and Stratakis, Ann NY Acad. Sci. 968: 256-270 (2002)). GPCRs activation of Gαs and Gαᵢ respectively increase and decrease intracellular cAMP levels (Hanoune and Defer, Annu Rev. Pharmacol. Toxical 42: 145-174 (2001)) (Hansom and Defr (2001)). The GTP-bound form of Gαₛ directly interacts and activates the 9 types of membrane-bound adenylyl cyclase (AC) known. Conversely, the GTP-bound form of Gαᵢ can directly interact and inhibit up to 6 different types of AC. ERK1/2 is activated by G_{q}, Gₛ and Gi-coupled GPCRs (Liebmann et al (1996); Pierce et al., Oncogene 20(13): 1532-1539 (2001); Gutkind, J. S., J. Biol Chem 273(4): 1839-42 (1998)) and, depending on the cellular context, several signaling pathways can be triggered to activate ERK1/2. Specifically, it is thought that Gᵢ-coupled GPCRs activate ERK1/2 mainly via the free (activated) Gβγ subunits (Crespo et al. Nature 369: 418-20 (1994); Faure et al., J. Biol Chem. 269(11): 7852-7854 (1999)) that recruit and activate soluble tyrosine kinases of the Src (Gutkind, 1998) and Bruton families (Wan et al., J. Biol Chem. 272(27): 17209-15 (1997)) or somehow transactivate receptor tyrosine kinases (RTKs) at the cell surface to initiate the cascade Liebmann et al. (2001); Wu et al. Bioch. Biophys Acta. 1582:100-106 (2002)).

In this earlier application, we showed that a rodent bitter receptor, mT2R05, the human, sweet taste receptor, hT1R2/hT1R3, and the human umami taste receptor, hT1R1/R3, couples to the activation of ERK1/2 and the inhibition of cAMP accumulation in HEK-293 cells, that the bitter substance cycloheximide, the sweeteners saccharin, sucrose, cyclamate, D-tryptophan and the savory amino acid MSG activate ERK1/2 exclusively in cells expressing their respective receptors. Collectively, our earlier results indicated that bitter compounds, sweeteners and monosodium glutamate (MSG) specifically activate their respective taste receptors to induce ERK1/2 activation and the reduction of cAMP accumulation in heterologous cells.

α-subunits of the Gᵢ family including Gαᵢ₁₋₁, Gαᵢ₁₋₂, Gαᵢ₁₋₃, Gαᵢ₀₋₁, Gαᵢ₀₋₂, α-transducin and α-gustducin contain a conserved carboxyl-terminal cysteine residue that is a site for modification by PTX, a 5′-diphosphate-ribosyltransferase isolated from Bortadella pertussis (Fields et al. Biochem J. 321(P1-3): 561-71 (1997)). PTX specifically and irreversibly modifies these G-protein subunits in vivo with attachment of an ADP-ribose moiety and, as a result, this covalent modification physically uncouples the G-protein from activation by GPCRs (Fields et al. (1997)). We further demonstrated that incubation of cells with PTX abolishes the activation of ERK1/2 by the bitter, sweet and umami taste receptors indicating that one or more members of the Gi family functionally link the taste receptors to this signaling pathway in HEK-293 cells, and further that taste GPCR we studied coupled to the inhibition of forskolin-induced cAMP accumulation in HEK-293 cells and that PTX-treatment totally abolishes the inhibition. These results clearly indicated that T1R and T2R taste receptors directly couple to one or more member of the Gαᵢ₁₋₃ subfamily in these cells. In this signaling pathway, activated Gαᵢ proteins directly interact and inhibit the membrane bound adenylyl cyclase.

Additionally, we showed that the sweet receptor clearly couples to a reduction of intracellular cAMP levels and activation of ERK1/2 through the direct functional coupling with Gᵢ. Our earlier results further clearly demonstrated that the umami receptor functionally couples to a reduction of intracellular cAMP levels and to the Gᵢ-induced activation of ERK1/2 in HEK-293 cells.

The results of this application corroborate our earlier results. Particularly, in another expression system we have shown that the activation of bitter (T2R) and sweet (T1R2/T1R3) taste receptors inhibits oCNGC activity and the resulting calcium influx in HEK-293 cells which co-express a human oCNGC and a functional T1R or T2R receptor (mT2R05 and hT1R2/hT1R3 exemplified). That this functional coupling is attributable to a G_{αi/o} protein is e.g., evidenced by the experimental results which indicate that (i) treatment of both mT2R05 and HT1R2/hT1R3 expressing HEK-293—oCNG cells with PTx prevented the isoproterenol-induced calcium influx by sweeteners and a bitter compound shown to activate these receptors; (ii) in the absence of PTx, the addition of isoproterenol elicited an increase in calcium influx cells, which calcium influx was inhibited in a dose-specific manner by the various sweeteners and bitter compound tested, and (iii) that the inhibition of isoprotenol-induced calcium reflux by taste modulatory compounds required the presence of a functional taste receptor activated by the particular taste modulator (e.g. a sweetener or bitter compound).

1. An assay for identifying a compound that modulates the activity of a T1R or T2R taste receptor comprising:
i. contacting a test cell that co-expresses (1) at least one functional T1R or T2R taste receptor, (2) a functional olfactory cyclic nucleotide gated channel (oCNGC) subunit, and (3) at least one G_{αi/o} protein that functionally couples to said T1R and T2R with a compound;
ii. detecting whether said compound modulates oCNGC activity; and
iii. identifying a compound as modulator of said functional taste if it results in a detectable change in intracellular calcium or sodium concentration relative to a control cell, which is identified as a cell that expresses oCNGC but not a T1R or T2R.

2. An assay for identifying whether a compound modulates the effect of another compound on T1R or T2R activity comprising:
i. contacting a test cell that co-expresses (1) at least one functional T1R or T2R taste receptor, (2) a functional olfactory cyclic nucleotide gated channel (oCNGC), and (3) at least one Gα_{i/o} protein that functionally couples to said T1R and T2R with a first compound known to activate said T1R or T2R;
ii. further contacting an equivalent test cell with said first compound and a candidate compound to be screened for its modulator effect on T1R or T2R activation induced by said first compound;
iii. evaluating the effect of said first compound on oCNGC activity;
iv. further evaluating the combined effect of said first compound and candidate compound on oCNGC activity; and
v. identifying the compounds that result in the oCNGC activity measured in (iv) to be significantly different than in (iii).

3. The assay of claim 1 or 2 which uses an oCNGC wherein at least one subunit has been modified resulting in a functional oCNGC that is more sensitive to cAMP.

4. The assay of claim 3 wherein said oCNGC comprises at least a modified oCNC1 subunit containing mutations which result in a oCNGC which is more sensitive to cAMP.

5. The assay of claim 4 wherein said mutation in said OCNC1 subunit comprise the change of a cysteine as position 458 to a tryptophan and the change of a glutamic acid at position 581 to a methionine.

6. The assay of claim 1 or 2 wherein oCNGC activity is detected based on whether there is a change in intracellular calcium or sodium concentration.

7. The assay of claim 6 wherein changes in intracellular calcium or sodium are detected by a fluorescence-based method.

8. The assay of claim 7 which comprises use of a fluorescent dye specific for calcium or sodium.

9. The assay of claim 8 wherein said dye is Fluo-4 or Fura-2.

10. The assay of claim 1 or 2 wherein oCNGC activity in the test cell and control cell are induced prior to contacting of said test cell and control cell with said candidate compound.

11. The assay of claim 10 wherein induction is effected by the addition of a compound that results in an increase in intracellular cAMP.

12. The assay of claim 11 wherein said compound activates adenylyl cyclase, guanylyl cyclase or phosphodiesterase inhibitor.

13. The assay of claim 11 wherein said compound is isoproterenol.

14. The assay of claim 1 or 2 wherein said test cell is selected from the group consisting of HEK, HEK-293, HEK-293T, COS, MDCK, BHK, NIH3T3, SWISS3T3 and CHO cells.

15. The assay of claim 1 or 2 wherein said test cell is selected from the group consisting of mammalian cells, amphibian cells, avian cells, bacterial cells, insect cells and yeast cells.

16. The assay of claim 15 wherein said test cell is a HEK-293 cell.

17. The assay of claim 1 or 2 wherein said taste receptor comprises at least one T2R.

18. The assay of claim 17 wherein said T2R is selected from the group consisting of mouse T2R, rat T2R, dog, T2R, cat T2R, monkey T2R and human T2R.

19. The assay of claim 18 wherein said T2R is a human T2R.

20. The assay of claim 18 wherein said T2R is a mouse T2R.

21. The assay of claim 18 wherein said T2R is a rat T2R.

22. The assay of claim 18 wherein said T2R is a dog T2R.

23. The assay of claim 18 wherein said T2R is a cat T2R.

24. The assay of claim 18 wherein said T2R is a monkey T2R.

25. The assay of claim 17 wherein said cell expresses a combination of different T2Rs.

26. The assay of claim 1 or 2 wherein said taste receptor comprises at least one T1R.

27. The assay of claim 26 wherein said T1R is selected from the group consisting of human, rat, mouse, dog, cat, or monkey T1R1, T1R2 and T1R3.

28. The assay of claim 27 wherein said cell co-expresses T1R1 and T1R3.

29. The assay of claim 27 wherein said T1R1 and T1R3 are human.

30. The assay of claim 27 wherein said T1R1 and T1R3 are mouse.

31. The assay of claim 27 wherein said T1R1 and T1R3 are rat.

32. The assay of claim 27 wherein said T1R1 and T1R3 are dog

33. The assay of claim 27 wherein said T1R1 and T1R3 are cat.

34. The assay of claim 27 wherein said T1R1 and T1R3 are monkey.

35. The assay of claim 27 wherein said cell co-expresses T1R2 and T1R3.

36. The assay of claim 35 wherein said T1R2 and T1R3 are human.

37. The assay of claim 35 wherein said T1R2 and T1R3 are mouse.

38. The assay of claim 35 wherein said T1R2 and T1R3 are rat

39. The assay of claim 35 wherein said T1R2 and T1R3 are dog.

40. The assay of claim 35 wherein said T1R2 and T1R3 are cat.

41. The assay of claim 35 wherein said T1R2 and T1R3 are monkey.

42. The assay of claim 1 or 2 wherein said oCNGC subunit is a human or rodent oCNGC subunit.

43. The assay of claim 42 wherein said oCNGC subunit is human.

44. The assay of claim 43 wherein said human oCNGC subunit is selected from the group consisting of oCNC1, oCNC2 and oCNCβ1b, and wherein said oCNGC subunit may comprise one or more modifications that yield an oCNGC that is more sensitive to cAMP.

45. The assay of claim 1 or 2, which comprises a high throughput, assay that screens a plurality of candidate compounds.

46. The assay of claim 1 or 2 wherein said test cells and control cells are seeded onto a multi-well test plate.

47. The assay of claim 1 or 2, which uses isolated test cell membranes.

48. The assay of claim 1 or 2 wherein changes in intracellular calcium concentrations are detected fluorimetrically using an automated imaging instrument.

49. The assay of claim 48 wherein said instrument is a fluorometric imaging plate reader (FLIPR).

50. The assay of claim 1 or 2 wherein changes in intracellular calcium concentrations are detected using fluorescence imaging microscopy.

51. The assay of claim 1 or 2 wherein said test cell stably expresses said functional T1R or T2R taste receptor.

52. The assay of claim 1 or 2 wherein said test cell transiently expresses said functional T1R or T2R taste receptor.

53. The assay of claim 1 or 2 which further comprises a control cell wherein a cell that expresses the identical oCNGC subunits and G_{αi/o} proteins is contacted but does not express same T1R or T2R taste receptor with said candidate compound to confirm that the effect of the candidate compound on oCNGC activity requires the co-expression of a functional taste receptor and a oCNG channel subunit.

54. The method of claim 1 or 2 wherein the test cell is an HEK-293 cell that stably expresses oCNC1 and oCNCβ1b.

55. The method of claim 54 wherein said test cell stably expresses mT2R05.

56. A cell that co-expresses at least one functional T1R or T2R taste receptor, at least one functional olfactory cyclic nucleotide gated channel (cCNGC) subunit and at least one G_{αi/o} protein.

57. The cell of claim 56 wherein said functional oCNGC comprises at least one modified oCNGC subunit that results in an oCNGC that is more sensitive to cAMP.

58. The cell of claim 57 wherein said modified oCNGC subunit is an oCNC1 subunit that comprises one or more mutations that enhance the sensitivity of the resultant oCNGC channel to cAMP.

59. The cell of claim 58 wherein said oCNC1 subunit comprises Cys458Trp and Glu581Met substitution modifications.

60. The cell of claim 54, which is selected from the group consisting of bacteria, yeast, worm, amphibian, insect, avian and mammalian cells.

61. The cell of claim 60 which is a mammalian cell.

62. The mammalian cell of claim 61, which is selected from the group consisting of HEK, HEK-293, HEK-293T, COS, MDCK, BHK, NIH3T3, SWISS3T3 and CHO cells.

63. The mammalian cell of claim 62 which is a HEK-293 cell.

64. The cell of claim 56 wherein the functional taste receptor comprises a T2R receptor polypeptide.

65. The cell of claim 56 wherein said functional taste receptor comprises at least one T1R receptor polypeptide.

66. The cell of claim 65, which co-expresses a T1R1 and T1R3 receptor polypeptides to produce a functional umami taste receptor.

67. The cell of claim 66 wherein said T1R1 and T1R3 are human T1R1 and T1R3.

68. The cell of claim 65 wherein said T1R1 and T1R3 comprise a mouse T1R1 and mouse T1R3.

69. The cell of claim 65 wherein said T1R1 and T1R3 comprise rat T1R1 and rat T1R3.

70. The cell of claim 65 wherein said T1R1 and T1R3 comprise dog T1R1 and dog T1R3.

71. The cell of claim 65 wherein said T1R1 and T1R3 comprise cat T1R1 and cat T1R3.

72. The cell of claim 65 wherein said T1R1 and T1R3 comprise monkey T1R1 and monkey T1R3.

73. The cell of claim 56 which co-expresses T1R2 and T1R3 receptor polypeptides to produce a functional sweet receptor.

74. The cell of claim 73 wherein said T1R2 and T1R3 receptor pplypeptides are human.

75. The cell of claim 73 wherein said T1R2 and T1R3 receptor polypeptides are rat.

76. The cell of claim 73 wherein said T1R2 and T1R3 polypeptides are mouse.

77. The cell of claim 73 wherein said T1R2 and T1R3 polypeptides are dog.

78. The cell of claim 73 wherein said T1R2 and T1R3 polypeptides are cat.

79. The cell of claim 73 wherein said T1R2 and T1R3 polypeptides are monkey.

80. The cell of claim 56 wherein the oCNGC subunits comprise human or rodent oCNGC subunits or functional variants thereof.

81. The cell of claim 80 wherein the oCNGC subunits are selected from the group consisting of OCNC1, OCNC2 and OCNCβ1b.

82. The cell of claim 81 which co-expresses human oCNC1 and oCNCβ1b or functional variants thereof.

83. The cell of claim 56 wherein said G_{αi/o} protein is selected from the group consisting of G_{αi-1}, G_{αi-2}, G_{αi-3}, G_{αo-1}, G_{αo-2}, G_{αz} or a variant or chimera that functionally couples said taste receptor.

84. The cell of claim 56 wherein said G_{αi/o} protein is a member of the G_{αi1-3} subfamily.

85. The cell of claim 56 wherein the T2R taste receptor is mouse T2R05.

86. A T1R or T2R modulator identified using an assay according to claim 1 or claim 2.

87. A composition suitable for human or animal consumption comprising a T1R or T2R agonist, antagonist, enhancer or modulator according to claim 86.

88. The assay of claim 1 or 2, which comprises confirming the effect of said compound on T1R or T2R mediated taste in human or animal taste tests.


SEQUENCE LISTING (13 sequences)

SEQ ID NO: 1 - protein, 239 aa, Homo sapiens
DEEYLFVIFDFLIGVLIFATIVGNVGSMISNMNATRAEFQAKIDAVKHYMQFRKVSKGMEAKVIRWFDYLWTNKKTVDEREILKNLPAKLRAEIAINVHLSTLKKVRIFHDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLD

SEQ ID NO: 2 - protein, 239 aa, Homo sapiens
EEEYLFMVGDFLLAVMGFATIMGSMSSVIYNMNTADAAFYPDHALVKKYMKLQHVNRKLERRVIDWYQHLQINKKMTNEVAILQHLPERLRAEVEVSVHLSTLSRVQIFQNCEASLLEELVLKLQPQTYSPGEYVCRKGDIGQEMYIIREGQLAVVADDGITQYAVLGAGLYFGEISIINIKGNMSGNRRTANIKSLGYSDLFCLSKEDLREVLSEYPQAQTIMEEKGREILLKMNKLD

SEQ ID NO: 3 - protein, 237 aa, Homo sapiens
LFEIVFQLLNYFTGVFAFSVMIGQMRDVVGAATAGQTYYRSCMDSTVKYMNFYKIPKSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKKARRMLRSNNKPK

SEQ ID NO: 4 - DNA, 14 bp, Artificial Sequence
  Description of Artificial Sequence Synthetic linker oligonucleotide
ggcgcgccgccatg

SEQ ID NO: 5 - protein, 239 aa, Rattus norvegicus
DEEYLFVIFDFLIGVLIFATIVGNVGSMISNMNATRAEFQAKIDAVKHYMQFRKVSKDMEAKVIKWFDYLWTNKKTVDEREVLKNLPAKLRAEIAINVHLSTLKKVRIFQDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLD

SEQ ID NO: 6 - protein, 239 aa, Rattus norvegicus
EEEYLFMVGDFLLAVMGFATIMGSMSSVIYNMNTADAAFYPDHALVKKYMKLQHVNKRLERRVIDWYQHLQINKKMTNEVAILQHLPERLRAEVAVSVHLSTLSRVQIFQNCEASLLEELVLKLQPQTYSPGEYVCRKGDIGREMYIIREGQLAVVADDGVTQYAVLGAGLYFGEISIINIKGNMSGNRRTANIKSLGYSDLFCLSKEDLREVLSEYPQAQAVMEEKGREILLKMNKLD

SEQ ID NO: 7 - protein, 237 aa, Rattus norvegicus
LFEIVFQLLNYFTGVFAFSVMIGQMRDVVGAATAGQTYYRSCMDSTVKYMNFYKIPRSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKAVLVTLKAGSVFGEISLLAVGGGNRRTANVVAHGFTNLFILDKKDLNEILVHYPESQKLLRKKARRMLRNNNKPK

SEQ ID NO: 8 - DNA, 1995 bp, Homo sapiens
atgaccgaaaaaaccaatggtgtgaagagctccccagccaataatcacaaccatcatgcacctcctgccatcaaggccaatggcaaagatgaccacaggacaagcagcaggccacactctgcagctgacgatgacacctcctcagaactgcagaggctggcagacgtggatgccccacagcagggaaggagtggcttccgcaggatagttcgcctggtggggatcatcagagaatgggccaacaagaatttccgagaggaggaacctaggcctgactcattcctcgagcgttttcgtgggcctgaactccagactgtgaccacacaggagggggatggcaaaggcgacaaggatggcgaggacaaaggcaccaagaagaaatttgaactatttgtcttggacccagctggggattggtactactgctggctatttgtcattgccatgcccgtcctttacaactggtgcctgctggtggccagagcctgcttcagtgacctacagaaaggctactacctggtgtggctggtgctggattatgtctcagatgtggtctacattgcggacctcttcatccgattgcgcacaggtttcctggagcaggggctgctggtcaaagataccaagaaactgcgagacaactacatccacaccctgcagttcaagctggatgtggcttccatcatccccactgacctgatctattttgctgtggacatccacagccctgaggtgcgcttcaaccgcctgctgcactttgcccgcatgtttgagttctttgaccggacagagacacgcaccaactaccctaacatcttccgcatcagcaaccttgtcctctacatcttggtcatcatccactggaatgcctgcatctattatgccatctccaaatccataggctttggggtcgacacctgggtttacccaaacatcactgaccctgagtatggctacctggctagggaatacatctattgcctttactggtccacactgactctcactaccattggggagacaccaccccctgtaaaggatgaggagtacctatttgtcatctttgacttcctgattggcgtcctcatctttgccaccatcgtgggaaatgtgggctccatgatctccaacatgaatgccacccgggcagagttccaggctaagatcgatgccgtgaaacactacatgcagttccgaaaggtcagcaaggggatggaagccaaggtcattaggtggtttgactacttgtggaccaataagaagacagtggatgagcgagaaattctcaagaatctgccagccaagctcagggctgagatagccatcaatgtccacttgtccacactcaagaaagtgcgcatcttccatgattgtgaggctggcctgctggtagagctggtactgaaactccgtcctcaggtcttcagtcctggggattacatttgccgcaaaggggacatcggcaaggagatgtacatcattaaggagggcaaactggcagtggtggctgatgatggtgtgactcagtatgctctgctgtcggctggaagctgctttggcgagatcagtatccttaacattaagggcagtaaaatgggcaatcgacgcacagctaatatccgcagcctgggctactcagatctcttctgcttgtccaaggatgatcttatggaagctgtgactgagtaccctgatgccaagaaagtcctagaagagaggggtcgggagatcctcatgaaggagggactgctggatgagaacgaagtggcaaccagcatggaggtcgacgtgcaggagaagctagggcagctggagaccaacatggaaaccttgtacactcgctttggccgcctgctggctgagtacacgggggcccagcagaagctcaagcagcgcatcacagttctggaaaccaagatgaaacagaacaatgaagatgactacctgtctgatgggatgaacagccctgagctggctgctgctgacgagccataa

SEQ ID NO: 9 - DNA, 1728 bp, Homo sapiens
atgagccaggacaccaaagtgaagacaacagagtccagtcccccagccccatccaaggccaggaagttgctgcctgtcctggacccatctggggattactactactggtggctgaacacaatggtcttcccagtcatgtataacctcatcatcctcgtgtgcagagcctgcttccccgacttgcagcacggttatctggtggcctggttggtgctggactacacgagtgacctgctatacctactagacatggtggtgcgcttccacacaggattcttggaacagggcatcctggtggtggacaagggtaggatctcgagtcgctacgttcgcacctggagtttcttcttggacctggcttccctgatgcccacagatgtggtctacgtgcggctgggcccgcacacacccaccctgaggctgaaccgctttctccgcgcgccccgcctcttcgaggccttcgaccgcacagagacccgcacagcttacccaaatgcctttcgcattgccaagctgatgctttacatttttgtcgtcatccattggaacagctgcctatactttgccctatcccggtacctgggcttcgggcgtgacgcatgggtgtacccggaccccgcgcagcctggctttgagcgcctgcggcgccagtacctctatagcttttacttctccacgctgatactgactacagtgggcgatacaccgccgccagccagggaagaagagtacctcttcatggtgggcgacttcctgctggccgtcatgggtttcgccaccatcatgggtagcatgagctctgtcatctacaacatgaacactgcagatgcggctttctacccagatcatgcactggtgaagaagtacatgaagctgcagcacgtcaaccgcaagctggagcggcgagttattgactggtatcagcacctgcagatcaacaagaagatgaccaacgaggtagccatcttacagcacttgcctgagcggctgcgggcagaagtggctgtgtctgtgcacctgtccactctgagccgggtgcagatctttcagaactgtgaggccagcctgctggaggagctggtgctgaagctgcagccccagacctactcaccaggtgaatatgtatgccgcaaaggagacattggccaagagatgtacatcatccgagagggtcaactggccgtggtggcagatgatggtatcacacagtatgctgtgctcggtgcagggctctactttggggagatcagcatcatcaacatcaaagggaacatgtctgggaaccgccgcacagccaacatcaagagcctaggttattcagacctattctgcctgagcaaggaggacctgcgggaggtgctgagcgagtatccacaagcacagaccatcatggaggagaaaggacgtgagatcctgctgaaaatgaacaagttggacgtgaatgctgaggcagctgagatcgccctgcaggaggccacagagtcccggctacgaggcctagaccagcagctggatgatctacagaccaagtttgctcgcctcctggctgagctggagtccagcgcacttaagattgcttaccgcattgaacggctggagtggcagactcgagagtggccaatgcccgaggacctggctgaggctgatgacgagggtgagcctgaggagggaacttccaaagatgaagagggcagggccagccaggagggacccccaggtccagagtga

SEQ ID NO: 10 - DNA, 2607 bp, Homo sapiens
atggctacaccatgtcagaacatggagctgaatcgcctagtccaggaccagatacctggccaggtggcctctgcctctgccctccaaaacctggtagagcatctccctaatgtgcccagctaccgcatcccaatcacccgcatccctgtcctcacctcccggagaaccagcttgtccaactccagcttcgccaaggagaccaggagctccatccgccaactagtgcctgccacgaaacagcacccagaagtgcaggtggaagatactgatgctgatagctgccccctcatggcagaagagaatccaccctcaaccgtgttgccgccaccgtctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataactacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagcccaaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaa

SEQ ID NO: 11 - protein, 7 aa, Artificial Sequence
  Description of Artificial Sequence Synthetic peptide
FKKSFKL

SEQ ID NO: 12 - protein, 13 aa, Artificial Sequence
  Description of Artificial Sequence Synthetic peptide
RRLIEDAEYAARG

SEQ ID NO: 13 - DNA, 11 bp, Artificial Sequence
  Description of Artificial Sequence Synthetic consensus sequence
ggggactttcc